(19) United States
(12) Patent Application Publication
(10) Pub. No.: US 2008/0262088 A1
(43) Pub. Date: Oct. 23, 2008

(54) METHODS, COMPOUNDS, AND COMPOSITIONS FOR TREATING METABOLIC DISORDERS AND DIABETES

(76) Inventors: Wendy HAUCK, Quebec (CA); Pavel Hamet, Quebec (CA)

Correspondence Address: MILLEN, WHITE, ZELANO & BRANIGAN PC, 2200 CLARENDON BLVD, STE 1400, ARLINGTON, VA, 22201, US

(21) Appl. No.: 11/963,038

(22) Filed: Dec. 21, 2007

Related U.S. Application Data
(60) Provisional application No. 60/916,488, filed on May 7, 2007.

(30) Foreign Application Priority Data
Dec. 22, 2006 (IB) PCT/IB2006/004262

(51) Int. Cl. A61K 31/10; A61K 31/155; A61K 31/64; A61P 3/00

(52) U.S. Cl. 514/592; 514/711; 514/635

(57) ABSTRACT

Disclosed herein are methods, compounds and compositions for preventing or treating a pancreatic disorder, including diabetes mellitus (e.g. type 1 and/or type 2 diabetes). The invention generally includes administering to a subject 1,3-propanedisulfonic acid or a pharmaceutically acceptable salt thereof, e.g., 1,3-propanedisulfonic acid sodium salt. The invention also relates to methods, compounds and compositions for improving or at least stabilizing pancreatic function(s) and for the prevention and/or treatment of metabolic syndrome and its components. The invention further relates to methods, compounds and compositions for the prevention and/or treatment of dyslipidemia, and more particularly for reducing levels of harmful serum lipid levels, especially cholesterol and triglycerides in patients in need thereof, including diabetic patients.

### RELATED APPLICATION

This application claims priority under 35 USC 119(e) from U.S. provisional application 60/916,488, filed May 7, 2007, and claims priority under 35 USC 365(a) to PCT/IB2006/004262, filed Dec. 22, 2006, both of which are incorporated herein by reference. This application is also related to U.S. patent application Ser. No. 11/643,946 filed Dec. 22, 2006, incorporated herein by reference.

### FIELD OF THE INVENTION

The invention also relates to methods, compounds, and compositions for preventing or treating renal disorder complications. The invention further relates to methods, compounds, and compositions for the prevention and/or treatment of dyslipidemias, a common complication of renal disorders, chronic kidney diseases, and nephropathy. The compounds, methods and compositions of the invention are also useful for the prevention or treatment of pancreatic disorders, diabetes, insulin resistance, metabolic disorders, including dyslipidemia and so-called metabolic syndrome, microvascular and macrovascular disorders and other conditions associated with diabetes. The invention further relates to methods, compounds, and compositions to restore or improve pancreatic function by preventing loss or stimulating neogenesis of islets of Langerhans and/or beta-cells and stabilizing the insulin secreting function of the pancreas.

### BACKGROUND OF THE INVENTION

Diabetes is caused by multiple factors and is characterized by elevated levels of plasma glucose (hyperglycemia) in the fasting state. There are two generally recognized forms of diabetes: type 1 diabetes, or insulin dependent diabetes, in which patients produce little or no insulin and type 2 diabetes, or noninsulin-dependent diabetes wherein patients produce insulin, while at the same time demonstrating hyperglycemia. Type 1 diabetes is typically treated with exogenous insulin administered via injection. However, type 2 diabetics often present “insulin resistance”, such that the effect of insulin in stimulating glucose and lipid metabolism in the main insulin-sensitive tissues, namely muscle, liver and adipose tissues, is diminished and hyperglycemia results.

Persistent or uncontrolled hyperglycemia that occurs in diabetes is associated with increased morbidity and premature mortality. Abnormal glucose homeostasis is also associated, both directly and indirectly, with obesity, hypertension and alterations in lipid, lipoprotein and apolipoprotein metabolism. Type 2 diabetics are at increased risk of cardiovascular complications such as atherosclerosis, coronary heart disease, stroke, peripheral vascular disease, hypertension, nephropathy, retinopathy and also neuropathy. Many patients who have insulin resistance, but have not developed type 2 diabetes, are also at risk of developing symptoms referred to as “Syndrome X”, or “Metabolic Syndrome”. Metabolic syndrome is characterized by insulin resistance, along with abdominal obesity, hyperinsulinemia, high blood pressure, low HDL (high density lipoproteins) and high VLDL (very low density lipoprotein), hypertriglyceridemia and hyperuricemia. Whether or not they develop overt diabetes, these patients are at increased risk of developing cardiovascular complications.

Current treatments for diabetes include drugs such as sulfonylureas or meglitinide which stimulate the pancreatic cells to produce more insulin and injection of insulin when these drugs become ineffective. However, dangerously low levels of plasma glucose can result and increased levels of insulin resistance can ultimately occur. Biguanidine's action relies on reduction of hepatic gluconeogenesis, decreased absorption of glucose from the gastrointestinal tract, and increased insulin sensitivity but it may cause unpleasant gastrointestinal side effects. Specific agents decreasing insulin sensitivity are Thiazolidinediones or TZDs which act by binding to PPARs (peroxisome proliferator-activated receptors), a group of receptor molecules inside the cell nucleus but TZDs have major side effects, including an increased prevalence of heart failure and weight gain. There is a continuing need for new methods of treating diabetes and related conditions.

Renal disorders involve an alteration in the normal physiology and function of the kidney. Renal disorders can result from a wide range of acute and chronic conditions and events, including physical, chemical, or biological injury, insult or trauma, disease such as, for example, hypertension, diabetes, congestive heart failure, lupus, sickle cell anemia, and various inflammatory and autoimmune diseases, HIV-associated nephropathies, etc. Renal disorders can lead to reduced kidney function, hypertension, and renal failure, seriously compromising quality of life, sometimes requiring dialysis and in certain circumstances, kidney transplantation.

Diabetic nephropathy also known as Kimmelstiel-Wilson syndrome and intercapillary glomerulonephritis, is a progressive kidney disease caused by angiopathy of capillaries in the kidney glomeruli. It is characterized by nodular glomerulosclerosis due to longstanding diabetes mellitus and is a prime cause for dialysis in many Western countries. The syndrome can be seen in patients with chronic diabetes. The disease is progressive and may cause death two or three years after the initial lesions and is more frequent in women. Diabetic nephropathy is the most common cause of chronic kidney failure and end-stage kidney disease in the United States. People with both type 1 and type 2 diabetes are at risk. The risk is higher if blood-glucose levels are poorly controlled. However, once nephropathy develops, the greatest rate of progression is seen in patients with poor control of their blood pressure.

Diabetic nephropathy is clinically well defined and is characterized by proteinuria, hypertension, edema and renal insufficiency. There are limited treatment options for diabetic nephropathy. Current treatments are primarily directed to improving complications of the diseases as follows: 1) control of blood-pressure (ACE-inhibitors inhibitors or Angiotensin receptor blockers (ARBs)); 2) Control of glycemic values; and 3) lipoproteic diet, exercise or other life styles modifications. However, there is an important need for better drugs and treatments since current treatment may have limited impact on the progressive decline in kidney function and patients still progress to renal replacement therapy, either dialysis or renal transplantation.

Hyperlipidemia is a major complication of diabetic nephropathy and is a determinant of progression of renal disorder in diabetes. Hyperlipidemia is a pathogenic factor for diabetic nephropathy and clinical studies involving therapeutic interventions for hyperlipidemia suggest the importance of this approach in at least slowing the progression of diabetic renal disorder (Rosario and Prabhakar (2006), Current Diabetes Reports, 6:455-462). Therefore, there is a need for methods and compounds for modulating blood lipids levels, and more particularly reducing levels of harmful serum lipid levels, especially cholesterol and triglycerides in diabetic patients.

Pancreatic islets of Langerhans are the only organ of insulin production in the body. However, they have a limited capacity for regeneration. This limited regeneration capacity, together with the susceptibility to apoptotic destruction, predisposes mammals to develop diabetes mellitus. Thus there is a need for products which can stimulate the regeneration or prevent apoptosis of islets of Langerhans to prevent or ameliorate the symptoms of diabetes mellitus. There is also a need for compounds and compositions for: (1) restoring beta-cell mass and function in an individual in need thereof; (2) preventing or treating type 1 diabetes in an individual in need thereof; (3) preventing or treating latent autoimmune diabetes of adults (LADA) in an individual in need thereof; (4) treating type 2 diabetes by preserving or increasing the number of functional insulin-producing cells (e.g., beta-cells) and/or (5) decreasing resistance to insulin and/or increasing insulin sensitivity.

### SUMMARY OF THE INVENTION

The invention relates to methods, compounds and compositions for the prevention and/or treatment of dyslipidemia, and more particularly for reducing serum levels of lipids involved in renal disorder complications, vascular and cardiovascular diseases, obesity and the like.

The invention further relates to methods, compounds and compositions for the prevention and/or treatment of hyperglycemia and more particularly for reducing serum levels of glucose involved in diabetes, obesity and the like.

In another aspect, this invention relates to a method for preventing or treating a renal disorder complication in a subject in need thereof comprising administering to said subject an effective amount of a compound of the Formula (I):

Y—(CH₂)ₙ—(CH)ₜ—[CH₂Y]ₘ (I)

wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2; wherein said subject does not have amyloidosis.

In another aspect, this invention relates to a method for the prevention or treatment of dyslipidemia in a subject in need thereof, comprising administering to said subject an effective amount of a compound of the Formula (I)

Y—(CH₂)ₙ—(CH)ₜ—[CH₂Y]ₘ (I)

wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2.

In another aspect, this invention relates to a method of reducing serum lipid levels in a subject in need thereof comprising administering to said subject an effective amount of a compound of the Formula (I):

Y—(CH₂)ₙ—(CH)ₜ—[CH₂Y]ₘ (I)

wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2.

In some embodiments, the invention pertains to methods and pharmaceutical compositions comprising the use of a therapeutically effective amount of a compound selected from the group consisting of 1,2-ethanedisulfonic acid, 1,2-ethanediol bis(hydrogen sulfate), 1,3-propanediol bis(hydrogen sulfate), 2-sulfomethyl-1,4-butanedisulfonic acid, and pharmaceutically acceptable salts thereof.

The invention also pertains to compounds, methods and compositions for the prevention and/or treatment of blood lipids-associated conditions by the administration of a compound of Formula (I) to a patient in need of such treatment.

In another aspect, this invention relates to a method for the prevention and/or treatment of a pancreatic disorder in a subject in need thereof comprising administering to said subject an effective amount of a compound of the Formula (I)

Y—(CH₂)ₙ—(CH)ₜ—[CH₂Y]ₘ (I)

wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2; wherein said subject does not have amyloidosis.

In preferred embodiments, administration of a compound of the Formula (I) has any of the following pharmaceutical effects: (i) increasing insulin levels circulating in blood in response to food, (ii) decreasing resistance to insulin and/or increasing insulin sensitivity in selected tissues (e.g. fat, muscle and liver), (iii) increasing insulin secretion by pancreatic cells, (iv) increasing beta-cells and/or islets of Langerhans neogenesis and/or regeneration of islets of Langerhans or preventing their destruction by apoptosis, (v) preventing apoptosis in beta-cells, and (vi) stabilizing, restoring, and/or improving pancreatic function, and more particularly stabilizing, restoring, and/or improving beta-cells size, growth and/or function.

In another aspect, this invention relates to a method for the prevention or treatment of hyperglycemia in a subject in need thereof, comprising administering to said subject an effective amount of a compound of the Formula (I) as defined hereinabove. In yet another embodiment, the invention includes a method for preventing or treating a disease that is directly related to an undesirably high glycemia or to undesirably low circulating levels of insulin and/or low insulin secretion by pancreatic cells and/or restoring its target organ sensitivity to its action on glucose disposal. In another related aspect, this invention relates to a method of reducing serum glucose levels in a subject in need thereof comprising administering to said subject an effective amount of a compound of the Formula (I). Preferably the disease is diabetes, e.g. type 1 and/or type 2. More preferably, the method includes administering to the subject a therapeutically effective amount of a compound of Formula (I), e.g., 1,3-propanedisulfonic acid or a pharmaceutically acceptable salt thereof, e.g. a disodium salt, such that renal function is stabilized or progression of the renal disorder is delayed.

In another aspect, an embodiment of the invention provides a method for treating a subject having diabetes, including administering to a subject in need thereof a compound or composition according to the invention, e.g., 1,3-propanedisulfonic acid or a pharmaceutically acceptable salt thereof.

The present invention relates to the production of islet cells and insulin in a subject by administering to the subject a compound or composition according to the invention, e.g., 1,3-propanedisulfonic acid or a pharmaceutically acceptable salt thereof.

The present invention also relates to methods for using a compound or composition according to the invention to produce new beta-cells and/or to prevent their destruction, for treating patients with diabetes mellitus. A related aspect of the invention concerns methods for the production of islet cells in the pancreas of a subject. Another related aspect of the invention concerns methods for the production of insulin in a subject by inducing the formation of functional beta-cells. Another related aspect of the invention concerns methods for the production of insulin in a subject by reducing beta-cell damage, apoptosis or death and/or by reducing islet malfunction. Another related aspect of the invention concerns methods for decreasing resistance to insulin and/or increasing insulin sensitivity in selected tissues (e.g. fat, muscle and liver). Yet another aspect of the invention relates to a method of treating diabetes in a patient in need of islet neogenesis, including administering to a subject in need thereof a compound or composition according to the invention, e.g., 1,3-propanedisulfonic acid or a pharmaceutically acceptable salt thereof.

In yet another aspect, the invention relates to a method of reducing insulin usage in an insulin-deficient diabetic patient, the method including administering compound(s) or composition(s) of the invention, e.g., 1,3-propanedisulfonic acid or a pharmaceutically acceptable salt thereof. In another aspect, the invention relates to a method for delaying the requirement for treating a diabetic patient with insulin by administering a therapeutically effective amount of a compound of the invention.

In another aspect, this invention relates to a method for the prevention and/or treatment of metabolic syndrome in a subject in need thereof, comprising administering to said subject an effective amount of a compound(s) or composition(s) of the invention, e.g., 1,3-propanedisulfonic acid or a pharmaceutically acceptable salt thereof.

In another aspect, this invention relates to a method for the prevention and/or treatment of diabetes with features of metabolic syndrome in a subject in need thereof, comprising administering to said subject an effective amount of a compound(s) or composition(s) of the invention, e.g., 1,3-propanedisulfonic acid or a pharmaceutically acceptable salt thereof.

In some embodiments, the method comprises administering a compound of the invention, e.g. 1,3-propanedisulfonic acid or a pharmaceutically acceptable salt thereof, and a second agent. In one aspect, the second agent is an anti-diabetic drug. In another aspect, the second agent is selected from biguanides and sulfonylureas, e.g. metformin or metformin with a sulfonylurea. In one embodiment, the method comprises administering a compound of the invention, allowing administration of a lower dose of the second agent than if administered alone, such that side effects are lowered. In another embodiment, an improved control of blood glucose level is achieved. In yet another embodiment, the method further provides treatment or prevention for one or more symptoms or features of metabolic syndrome.

In some embodiments, the present invention concerns the above-identified methods in subjects other than those having amyloidosis (e.g. AA amyloidosis, IAPP-related amyloidosis) and/or other than those having a nephropathy (e.g. diabetic nephropathy).

In one aspect, the invention relates to the treatment of diabetes by administering a compound of Formula I. In one embodiment, such treatment does not include treating a diabetic patient in which a compound of Formula I is being used in nephrology. In another embodiment, such treatment does not include treating a diabetic patient to whom a compound of Formula I is being administered for the purpose of treating a renal disorder. In another embodiment, the diabetic patient is not otherwise undergoing treatment with a compound of Formula I for any purpose, e.g., for treatment of renal disorders, nephropathy, or amyloidosis, etc. In yet another embodiment, such treatment includes treating diabetes in a diabetic patient as a consequence of treating said patient with a compound of formula I for the purpose of treating a renal disorder, e.g. nephropathy.

These and other objects, advantages and features of the present invention will become apparent to those persons skilled in the art upon reading the details of the invention more fully set forth below.

### DESCRIPTION OF DRAWINGS

Asterisk (*) in Figures are used to show the results in the specific Figure where the difference between treated and control rats is statistically significant.

FIG. 1 is a line graph showing daily dose of 1,3-propanedisulfonic acid administered to Zucker diabetic obese male rats over a period of 12 weeks, according to Example 11(a).

FIG. 2A is a line graph showing median serum creatinine for control and treated satient Zucker diabetic obese male rats, over a period of 12 weeks, according to Example 11(a).

FIG. 2B is a line graph showing median body weight corrected creatinine clearance for control and treated satient Zucker diabetic obese male rats over a period of 12 weeks, according to Example 11(a).

FIG. 3 is a line graph showing median urine protein content for control and treated satient Zucker diabetic obese male rats, over a period of 12 weeks, according to Example 11(a).

FIG. 4A is a line graph showing median serum uric acid in control and treated satient Zucker diabetic obese male rats, over a period of 12 weeks, according to Example 11(a).

FIG. 4B is a line graph showing median body weight corrected uric acid clearance in control and treated satient Zucker diabetic obese male rats over a period of 12 weeks, according to Example 11(a).

FIG. 5 is a line graph showing median serum potassium levels (kalemia) in control and treated satient Zucker diabetic obese male rats, over a period of 12 weeks, according to Example 11(a).

FIG. 6A is a line graph showing median serum triglycerides in control and treated satient Zucker diabetic obese male rats, over a period of 12 weeks, according to Example 11(a).

FIG. 6B is a bar graph showing median serum cholesterol for control and treated satient Zucker diabetic obese male rats at week 10 and week 12, according to Example 11(a).

FIG. 7 is a bar graph showing glomerular global score for control and treated satient Zucker diabetic obese male rats after 12 weeks, according to Example 11(a). Categories: (−) no global score; (±) mild global score; (+) mild to moderate global score; (++) moderate global score.

FIG. 8 is a box graph showing distribution of satient serum insulin, as measured by RIA, for control and treated Zucker diabetic obese male rats after 12 weeks, according to Example 11(b). Box plot lower limit represents the 25^{th} percentile, whereas higher limit represents 75^{th} percentile. Whiskers above and below indicate 90^{th} and 10^{th} percentiles, and median and mean values are represented by solid and dashed lines respectively.

FIG. 9A is a line graph showing mean (±SEM) serum glucose levels (Hexokinase (HK) II method) in control and treated satient Zucker diabetic obese male rats, over a period of 12 weeks, according to Example 11(b).

FIG. 9B is a line graph showing median capillary blood glucose levels (glucose meter kit) in control and treated satient Zucker diabetic obese male rats, over a period of 12 weeks, according to Example 11(b).

FIG. 10 is a line graph showing median diuresis in control and treated satient Zucker diabetic obese male rats, over a period of 12 weeks, according to Example 11(b).

FIG. 11 is a box graph showing distribution of number of islets of Langerhans per field counted during histology of the pancreas from control and treated Zucker diabetic obese male rats at week 12, according to Example 11(b). Box plot lower limit represents the 25^{th} percentile, whereas higher limit represents 75^{th} percentile. Whiskers above and below indicate 90^{th} and 10^{th} percentiles, and median and mean values are represented by solid and dashed lines respectively.

### DETAILED DESCRIPTION OF THE INVENTION

The term “renal disorder”, “renal disease” or “kidney disease” means any alteration in normal physiology and function of the kidney. This can result from a wide range of acute and chronic conditions and events, including physical, chemical or biological injury, insult, trauma or disease, such as for example hypertension, diabetes, congestive heart failure, lupus, sickle cell anemia and various inflammatory, infectious and autoimmune diseases, HIV-associated nephropathies etc. This term includes but is not limited to diseases and conditions such as kidney transplant, nephropathy; chronic kidney disease (CKD); Glomerulonephritis; inherited diseases such as polycystic kidney disease; nephromegaly (extreme hypertrophy of one or both kidneys); nephrotic syndrome; end stage renal disease (ESRD); acute and chronic renal failure; interstitial disease; nephritis; sclerosis, an induration or hardening of tissues and/or vessels resulting from causes that include, for example, inflammation due to disease or injury; renal fibrosis and scarring; renal-associated proliferative disorders; and other primary or secondary nephrogenic conditions. Fibrosis associated with dialysis following kidney failure and catheter placement, e.g., peritoneal and vascular access fibrosis, is also included.

Renal disorders or kidney diseases may also be generally defined as a “nephropathy” or “nephropathies”. The terms “nephropathy” or “nephropathies” encompass all clinical-pathological changes in the kidney which may result in kidney fibrosis and/or glomerular diseases (e.g. glomerulosclerosis, glomerulonephritis) and/or chronic renal insufficiency, and can cause end stage renal disease and/or renal failure. Some aspects of the present invention relate to compositions and their uses for the prevention and/or treatment of hypertensive nephropathy, diabetic nephropathy, and other types of nephropathy such as analgesic nephropathy, immune-mediated glomerulopathies (e.g. IgA nephropathy or Berger's disease, lupus nephritis), ischemic nephropathy, HIV-associated nephropathy, membranous nephropathy, glomerulonephritis, glomerulosclerosis, radiocontrast media-induced nephropathy, toxic nephropathy, analgesic-induced nephrotoxicity, cisplatin nephropathy, transplant nephropathy, and other forms of glomerular abnormality or injury; glomerular capillary injury (tubular fibrosis). In some embodiments, the terms “nephropathy” or “nephropathies” refers specifically to a disorder or disease where there is either the presence of proteins (i.e. proteinuria) in the urine of a subject and/or the presence of renal insufficiency.

The term “fibrosis” refers to abnormal processing of fibrous tissue, or fibroid or fibrous degeneration. Fibrosis can result from various injuries or diseases, and can often result from chronic transplant rejection relating to the transplantation of various organs. Fibrosis typically involves the abnormal production, accumulation, or deposition of extracellular matrix components, including overproduction and increased deposition of, for example, collagen and fibronectin. As used herein, the terms “kidney fibrosis” or “renal fibrosis” or “fibrosis of the kidney” refer to diseases or disorders associated with the overproduction or abnormal deposition of extracellular matrix components, particularly collagen, leading to the degradation or impairment of kidney function.

By “pancreas” is meant the large, elongated, racemose gland situated transversely behind the stomach, between the spleen and the duodenum. The pancreas is composed of an endocrine portion (the pars endocrina) and an exocrine portion (the pars exocrina). The pars endocrina, which contains the islets of Langerhans, produces and secretes proteins, including insulin, directly into the blood stream. The pars exocrina contains secretory units and produces and secretes a pancreatic juice, which contains enzymes essential to protein digestion, into the duodenum.

By “islet cell” is meant a cell having a phenotype similar to the hormone-producing cells normally comprising the pancreatic islets of Langerhans, and generally characterized by the expression of markers that normally distinguishing the cells in the pancreatic islets of Langerhans from other pancreatic cells, such as insulin, glucagon, somatostatin, pancreatic polypeptide, or islet amyloid polypeptide (IAPP or amylin).

By “beta-cell”, or “β-cell” is meant a pancreatic islet cell having a phenotype characterized by the expression of markers that normally distinguish the beta-cells from the other pancreatic islets cells, such as insulin, Nkx6.1 or glucokinase. The term “pancreatic disorder”, “pancreatic disease” or “beta-cell related disease” means any alteration in normal physiology and/or function of the pancreas. As used herein, it more particularly refers to the endocrine function of the pancreas which relates to the production and/or secretion of insulin and maintenance of appropriate blood glucose levels. These terms also encompass all clinical-pathological conditions or diseases that are directly or indirectly related to an undesirably high glycemia or undesirably low levels of blood insulin. This can result from a wide range of acute and chronic conditions and events, including physical, chemical or biological injury, insult, trauma or disease, such as for example type 1 diabetes, type 2 diabetes, maturity-onset diabetes of the young, latent autoimmune diabetes of adults (LADA), gestational diabetes, obesity, hypertension, metabolic syndrome, renal disorders, etc. The terms “pancreatic disorder”, “pancreatic disease” or “beta-cell related disease” also include but are not limited to diseases and conditions where preventing loss or stimulating neogenesis of islets of Langerhans and/or beta-cells, stabilizing the insulin secreting function of the pancreas would be desirable (e.g., type 1 and type 2 diabetes). The compounds and compositions of the invention are useful for preventing or treating diabetic nephropathy in a subject in need thereof. Diabetic nephropathy is a clinically well-defined pathology characterized by proteinuria, hypertension, edema and renal insufficiency. Characteristic aspects of diabetic nephropathy include glomerulosclerosis, modification of the vascular structure, and tubulointerstitial disease. The first clinical evidence of diabetic nephropathy is often the presence of albuminuria in the urine, e.g. microalbuminuria or macroalbuminuria.

As is known, diabetic nephropathy is typically characterized by the following: 1) glomerulosclerosis, 2) modification of the vascular structure, mainly in the small arterioles and 3) tubulointerstitial disease. The most characteristic aspect of diabetic nephropathy is the glomerular injury, detectable by the enlargement of the mesangium and by the thickening of the basal membrane, which often looks like a diffuse cicatrisation of the whole glomerule. The first clinical evidence of diabetic nephropathy is the presence of albuminuria or proteinuria. One refers to microalbuminuria when the amount of albumin in the urine is less than or equal to <300 mg/day and proteinuria when the tolal amount of protein in the urine is greater than 1 g/day. Prevention, reduction or elimination of symptoms or complications of HIV-associated nephropathy in the context of the present invention refers to: prevention of HIV-associated nephropathy before it occurs (for example if the treatment begins with the manifestation of initial clinical indications of HIV such as decrease in CD4-bearing cells), elimination of established HIVAN altogether (as determined, for example, by the return of renal functions parameters to normal), or reduction in the undesired symptoms of the disease manifested by the decrease in the severity of an existing condition of HIVAN. The reduction in the undesired symptoms may be determined for example by the improvement in renal function as compared to the function prior to treatment. Such remediation may be evident in a delay in the onset of renal failure (including dialysis or transplant) or in a decrease in the rate of the deterioration of renal functions as determined for example by the slowing of the rate of the increase of proteinuria or slowing the rate of the rise in serum creatinine or by the fall in the parameter of creatinine clearance or GFR), or decrease in at least one symptom or complication caused by HIVAN including hospitalization rate or mortality.

The present invention further relates to methods, compounds and compositions for preventing and/or treating a renal disorder complication. The term “renal disorder complication” refers to a secondary condition correlated with a renal disorder, a health condition, an accident, or a negative reaction occurring during the course of a renal disorder that can become worse in its severity. A “renal disorder complication” is usually associated with increasing severity of the renal disease in the subjects suffering from symptoms or pathological changes, which can become widespread throughout the body or affecting other organ systems. As used herein, the term “renal disorder complication” encompasses, but is not limited to vascular diseases (e.g. hypertension, macrovascular complications, microvascular complications, etc.), cardiovascular diseases (e.g. arteriosclerosis, atherosclerosis, coronary artery disease, congestive heart failure, stroke, angina, ischemic heat disease, myocardial infarction, etc), diabetic dyslipidemia, hyperlipidemia (e.g. hypercholesterolemia, hypertriglyceridemia, hyperlipoproteinemia), metabolic syndrome, obesity, anemia, edema, pancreatitis, weak bones, poor nutritional health and nerve damage.

The present invention further relates to methods, compounds and compositions for the prevention and/or treatment of dyslipidemias. The term “dyslipidemias” or “dyslipidemia” encompass all clinical-pathological conditions or diseases that are directly or indirectly related to undesirably high or low levels, and/or undesirable ratios, of any circulating blood lipids and/or lipoproteins, including but not limited to levels and/or ratios of triglycerides, cholesterol, ApoB, LpA, high density lipoprotein (HDL), high-density lipoprotein cholesterol (HDLC), very low density lipoprotein cholesterol (VLDLC), low density lipoprotein cholesterol (LDLC), intermediate density lipoprotein cholesterol, low density lipoprotein (LDL), and free fatty acids.

The term dyslipidemia encompasses disorders of lipoprotein metabolism, including lipoprotein overproduction or deficiency, hyperlipidemia (e.g. hypercholesterolemia, hypertriglyceridemia, hyperlipoproteinemia, etc), diabetic dyslipidemia, and also other diseases and conditions wherein blood lipids levels are considered a pathogenic factor, including, but not limited to: vascular diseases (e.g. hypertension, macrovascular complications, microvascular complications, etc.), cardiovascular diseases (e.g. arteriosclerosis, atherosclerosis, coronary artery disease, congestive heart failure, stroke, angina, ischemic heat disease, myocardial infarction, etc), metabolic syndrome, and obesity.

In another aspect, the compounds are useful in preventing or treating nephropathies (e.g., diabetic nephropathy). The methods generally include administering to a subject a compound of the present invention as described herein. For example, in one embodiment, the compound is 1,3-propanedisulfonic acid or a pharmaceutically acceptable salt thereof. In one embodiment, the nephropathy is diabetic nephropathy. In one embodiment, administration of a compound of the invention may result in improved kidney function. In one embodiment, administration of a compound of the invention may result in the lowering the urinary excretion of albumin. In another embodiment, administration of a compound of the invention may result in increased creatinine clearance and/or uric acid clearance.

The invention also concerns methods, compounds and pharmaceutical compositions for the prevention and/or treatment (including reversal and cure) of mammals (including humans and animals) suffering from a pancreatic disorder. More particularly, the methods, compounds and compositions herein are useful for the prevention and/or treatment of humans suffering of a disease or condition caused by, or associated with diabetes mellitus (e.g. type 1, type 2, LADA, maturity-onset diabetes of the young, adult-onset diabetes and gestational diabetes), hyperglycemia, insulin insufficiency, beta-cell insufficiency or the like where there is insufficient insulin to maintain blood glucose levels (e.g. pancreatic exhaustion)). In some embodiments, administration of a compound of the invention may result in improved pancreatic function. In some embodiments, the present invention concerns the prevention and/or treatment of pancreatic disorders in subjects other than those having amyloidosis (e.g. AA amyloidosis, IAPP-related amyloidosis) and/or other than those having a nephropathy (e.g. diabetic nephropathy or insulin resistance).

In some embodiments the present invention concerns the treatment of patients with insulin dependent diabetes mellitus (i.e. type I or IDDM).

In some embodiments, the present invention concerns the treatment of patients with non-insulin diabetes mellitus (i.e. type II or NI-DDM).

The method of the present invention comprises administering to a mammal, e.g., a human patient or animal in need thereof, a preventative-or therapeutically-effective amount of a compound or pharmaceutical composition as defined herein.

Most insulin dependent diabetic patients require insulin injection at least on a daily basis. Multiple doses per day of insulin are currently recommended to achieve an adequate control of the disease, and the insulin administration is indicated by results of frequent glucose monitoring, another activity which is required of a diabetes patient for optimal management of the disease, which is performed for example as often as five times daily. In yet another aspect, the invention relates to a method of reducing insulin usage in an insulin-deficient diabetic patient, the method including administering compound(s) or composition(s) of the invention. According to that embodiment, as a result of this administration, remission of diabetes is initiated, so that the standard dosage of insulin given to a diabetic patient prior to therapy is reduced, as determined by the level of blood glucose obtained by monitoring, for example, by self-monitoring by the patient, during and following treatment. Remission from diabetes due to successful treatment according to the invention may be indicated by a decreased fasting blood level of glucose, and by a decreased level and duration of elevated blood glucose in response to a dietary challenge of sugar consumption. In yet another related aspect, the invention relates to a method of improving insulin sensitivity and/or decreasing insulin resistance in a subject in need of insulin, the method including administering compound(s) or composition(s) of the invention. Thus, in a preferred embodiment, insulin delivery after administering the compound(s) or composition(s) of the invention is reduced to less that about 75%, or to less that about 50%, or to less that about 10% or to less that about 1%, compared to usage in the diabetic patient before administration of the compound(s) or composition(s) of the invention. In other preferred embodiments, insulin administration is reduced from, for example, five injections to two injections per day; from two injections to one injection per day; and from one to none, as indicated by data obtained from monitoring blood glucose levels.

In some embodiment, the methods of the invention further comprise the step of evaluating the subject for one or more of the following parameters: (1) insulin blood levels; (2) glucose blood levels; (3) body weight. For instance, in one embodiment, the method comprises monitoring the blood glucose level at intervals of about once per day or less than about once per day; and reiterating administering the composition to the patient with a dosage adjusted according to the patient's blood glucose level. One of ordinary skill in the art of pharmacology, when treating a diabetic patient, is familiar with adjusting insulin dosage to levels of blood glucose following fasting and under other physiological conditions.

In another aspect, this invention relates to a method for improving insulin sensitivity and/or decreasing insulin resistance in a subject, the method including administering compound(s) or composition(s) of the invention.

In another aspect, this invention relates to a method for controlling or reducing hyperkalemia in a subject in need thereof, comprising administering to said subject an effective amount of a compound(s) or composition(s) of the invention, e.g., 1,3-propanedisulfonic acid or a pharmaceutically acceptable salt thereof. In one embodiment, the administration of the compound(s) or composition(s) of the invention increase potassium excretion.

In another aspect, this invention relates to a method for controlling, alleviating or reducing cardiovascular complications in a subject in need thereof, comprising administering to said subject an effective amount of a compound(s) or composition(s) of the invention, e.g., 1,3-propanedisulfonic acid or a pharmaceutically acceptable salt thereof. In one embodiment, the administration of the compound(s) or composition(s) of the invention increase uric acid excretion and/or lower uric acid in serum.

In preferred embodiments, 1,3-propanedisulfonic acid and/or 1,3-propanedisulfonic acid sodium salt is administered to the subject. Other suitable salts include, but are not limited to lithium, potassium, calcium, magnesium, mesylate, trialkylammonium and aluminum salts.

The term “subject” includes living organisms in which renal disorders or nephropathy can occur, or which are susceptible to kidney disorder or nephropathy. The term “subject” includes animals (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as chickens, ducks, Peking ducks, geese, and transgenic species thereof. Preferably, the subject is a mammal. More preferably, the subject is a human.

In some embodiments, the subject is a human patient having or susceptible of having glomerular filtration problems (e.g. diabetic nephropathy) and/or a renal failure. In some embodiments, the subject is a human patient having or susceptible to have a dyslipidemia, including but not limited to diabetic dyslipidemia, hyperlipidemia, vascular and cardiovascular diseases, metabolic syndrome X, and obesity.

In some embodiments, the subject may be suffering from a disorder such as, for example, diabetes, HIV, advanced progressive renal disease, and fibrotic renal disease and/or any of the diseases/disorders described herein. In one aspect the subject does not have amyloidosis. In one aspect the subject does not have Amyloid A (AA) amyloidosis. In another embodiment, the subject does have amyloidosis. In another embodiment, the subject does have Amyloid A (AA) amyloidosis.

In some embodiments the renal disease is not related to amyloid and the subject may or may not have amyloidosis (e.g. AA amyloidosis or IAPP-related amyloidosis). In some embodiments the nephropathy is not related to amyloid and the subject may or may not have amyloidosis (e.g. AA amyloidosis or IAPP-related amyloidosis). In some embodiments the diabetic nephropathy is not related to amyloid and the subject may or may not have amyloidosis (e.g. AA amyloidosis or IAPP-related amyloidosis). In some embodiments the renal disorder complication is not related to amyloid and the subject may or may not have amyloidosis (e.g. AA amyloidosis or IAPP-related amyloidosis). In a particular embodiment, in all the methods of this invention, the subject does not have amyloidosis (e.g. AA amyloidosis or IAPP-related amyloidosis). In a particular embodiment, in all the methods of this invention, the subject does not have AA amyloidosis. In a particular embodiment, in all the methods of this invention, the subject does not have IAPP-related amyloidosis. In some embodiments, the subject may be exhibiting proteinuria (e.g. microalbuminuria or macroalbuminuria). In some embodiments, the subject may have kidneys that have become less able to clear toxins from the blood, such as urea, uric acid and creatinine. In some embodiments, the methods, compounds or compositions of the invention are effective in slowing the decline in a patient's creatinine clearance by at least 0.5, 1, 2, 5, 10, 15, or 20 ml/min/1.73 m²/year. In some embodiments, the methods, compounds or compositions of the invention are effective in stabilizing a patient's uric acid clearance by at least 1, 2, 5, 10, 15 or 20 mg/dL.

In some embodiments, the subject is at risk of, or has been diagnosed with, nephropathy, e.g. diabetic nephropathy. Typically a normal glomerular filtration rate (GFR) in humans is from about 100 to about 140 ml/min. In some embodiments, the subject is a human patient having advanced nephropathy (i.e. a GFR of under 75 ml/min). In some embodiments, the subject is a human patient having ESRD (i.e. GFR of less than 10 ml/min). In some embodiments, the methods, compounds or compositions of the invention are effective in increasing the patients' GFR value by at least 1, 5, 10, 15, 20 or 25, ml/min or more.

In some embodiments, the subject is at risk of, or has been diagnosed with, a kidney disease. In various embodiments, the subject is a human patient having or progressing towards stage I kidney disease, stage II kidney disease, stage III kidney disease, stage IV kidney disease or stage V kidney disease. In some embodiments, the methods, compounds or compositions of the invention are effective in stabilizing or in improving the patient's kidney disease ((e.g. from stage V to stage IV, or from stage IV to stage II, or from stage III to stage II, or from stage II to stage I).

In some embodiments, the subject is at risk of, or has been diagnosed with, proteinuria. In some embodiments, the subject is a human patient producing less than about 300 mg/day of protein in its urine. In some embodiments, the subject is a human patient producing more that about 1 g/day of protein in its urine. In some embodiments, the subject is a human patient having microalbuminuria. In some embodiments, the subject is a human patient with albumin amount in the urine exceeds 200 μg/min. In some embodiments, the methods, compounds or compositions of the invention are effective in lowering the patient's albuminuria by at least 10, 25, 50, 75, 100, 150, 200 μg/min or more.

In some embodiments, the subject is at risk of, or has been diagnosed with hyperkalemia. Normal potassium levels in human blood is 3.5-5.0 mEq/L. Typically, hyperkalemia is defined by potassium levels greater than 5.5 mEq/L. In some embodiments, the subject is a human patient having mild hyperkalemia, i.e. having potassium levels of about 5.5 to about 6.0 mEq/L. In some embodiments, the subject is a human patient having moderate hyperkalemia, i.e. having potassium levels of about 6.1 to about 7.0 mEq/L. In some embodiments, the subject is a human patient having severe hyperkalemia, i.e. having potassium levels of about 7.0 mEq/L and greater. In some embodiments, the methods, compounds or compositions of the invention are effective in decreasing the patient's potassium levels by at least 0.25, 0.5, 0.75, 1.0, 1.25, 1.5, 1.75, 2.0 mEq/L or more.

In some embodiments, the subject is at risk of, or has been diagnosed with, hypertension or high blood pressure. There is often a strong correlation between hypertension and kidney diseases such as nephropathy, particularly diabetic nephropathy. Individuals with poor kidney function frequently exhibit hypertension. In some embodiments, the subject is a hypertensive human patient having a systolic pressure of 140 mm Hg or higher and/or a diastolic pressure of 90 mm Hg or higher. In some embodiments, the subject is a prehypertensive human patient having a systolic pressure of about 120-139 mm Hg or higher and/or a diastolic pressure of 80-89 mm Hg or higher. In some embodiments, the methods, compounds or compositions of the invention are effective in lowering the patients' systolic and/or diastolic blood pressure by at least 1, 2, 3, 4, 5, 6, 7, 8, 9, 10 mm Hg or more.

In some embodiments, the subject is a hyperlipidemic human patient. In some embodiments, the levels of lipids in the blood are too high, and the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (see http://www.americanheart.org/ and http://www.nhlbi.nih.gov/health/public/heart/, respectively). In some embodiments, the subject is a hypercholesterolimic human patient having a plasma LDL cholesterol level over than 100 mg/dL and/or a plasma HDL cholesterol level of 40 mg/dL or lower. In some embodiments, the subject is a hypertriglycemic human patient having borderline-high plasma triglycerides level of 150 to 199 mg/dL, or high plasma triglycerides level of 200 to 499 mg/dL, or very high plasma triglycerides level of 500 mg/dL or higher. Those levels are based on measurement under fasting conditions. Elevated triglycerides are frequently found in association with kidney diseases and nephropathy, particularly diabetic nephropathy. In some embodiments, the methods, compounds or compositions of the invention are effective in lowering the patient's LDL cholesterol level and/or plasma triglycerides level by at least 5, 10, 15, 20, 30, 40, 50, 75, 100, 125, 150, 175, 200 mg/dL or more. In some embodiments, the methods, compounds or compositions of the invention are effective in increasing the patient's HDL cholesterol level and/or plasma triglycerides level by at least 1, 2, 5, 10, 15, 20, 25, 30 mg/dL or more. An example of successive treatment of hypercholesterolemia according to the invention is aimed at lowering human serum cholesterol levels to under 5.0 mmol/l.

In some embodiments, the subject is overweight or obese. In some embodiments, the subject is an obese human patient having a body mass index (BMI) of about 25 to 30 (grade 1), or a BMI of 30-40 (grade 2), or a BMI of over 40 (grade 3). In some embodiments, the methods, compounds or compositions of the invention are effective in reducing the patient's body mass index of a value of 1, 2, 5, 10, 15, 20, 25, 30, 35, 40 or more. In some embodiments, the methods, compounds or compositions of the invention are effective in improving the patient's BMI grade (e.g. from grade 3 to grade 2, or from grade 2 to grade 1).

In some embodiments, the subject is at risk of or has been diagnosed with metabolic syndrome (also known under various names such as syndrome X, insulin resistance syndrome, Reaven's syndrome and CHAOS). In some embodiments, the subject is an human patient with presence of three or more of these components: elevated serum triglycerides (e.g. over 150 mg/dL), low HDL (e.g. under 40 mg/dl for men and under 50 mg/dl for women), central obesity (i.e. increased waist circumference: over 102 cm in males and over 88 cm in females), Elevated blood pressure, and high fasting plasma glucose (e.g. 100 mg/dl). In some embodiments, the methods, compounds or compositions of the invention are effective in losing any one of the above mentioned components of metabolic syndrome. Associated diseases and signs of metabolic syndrome are fatty liver (especially in concurrent obesity), progressing to non-alcoholic fatty liver disease, polycystic ovarian syndrome, hemochromatosis (iron overload); and acanthosis nigricans (a skin condition featuring dark patches). In some embodiments, the methods, compounds or compositions of the invention are effective in the prevention and/or treatment of any of those associated diseases.

In some embodiments, the subject is at risk of or has been diagnosed with diabetes (e.g. type 1, type 2, maturity-onset diabetes of the young, latent autoimmune diabetes of adults (LADA), gestational diabetes). In some embodiments, the compound(s) or composition(s) of the invention is administered in the early stages of onset of clinical symptoms of diabetes.

In some embodiments, the subject is hyperglycemic. In some embodiments, the subject's blood glucose levels are elevated, and the compound(s) and/or composition(s) of the invention are administered to a patient to restore normal levels. Normal levels of glucose are reported in medical treatises known to those of skill in the art. Typically blood sugar level is measured by means of a glucose meter, with the result either in mg/dL (milligrams per deciliter in the USA) or mmol/L (millimoles per litre in Canada and Europe) of blood. For example, the average normal person has a glucose level of around 4.5 to 7.0 mmol/L (80 to 125 mg/dL). In the diabetic patient a before-meal level of <6.1 mmol/L (<110 mg/dL) and a level two hours after the start of a meal of <7.8 mmol/L (<140 mg/dL) is acceptable. In some embodiments according to the invention, the subject blood glucose levels are above 150 mg/dl, or 175 mg/dl, or 200 mg/dl, or 225 mg/dl, or above 250 mg/dl, or over 300 mg/dl.

In some embodiments, the subject is a human patient with type 2 diabetes. As is known, type 2 diabetes results from a combination of insulin resistance and impaired insulin secretion, but ultimately many people with type 2 diabetes show markedly reduced pancreatic beta-cell mass and function which, in turn, causes type 2 diabetic patients to have a “relative” deficiency of insulin because pancreatic beta-cells are producing insufficient insulin to adequately allow glucose into cells to produce energy. Uncontrolled type 2 diabetes leads to excess glucose in the blood, resulting in hyperglycemia, or high blood sugar. A person with type 2 diabetes experiences fatigue, increased thirst, frequent urination, dry, itchy skin, blurred vision, slow healing cuts or sores, more infections than usual, numbness and tingling in feet. In some embodiments, the methods, compounds or compositions of the invention are effective in improving, curing and/or alleviating one or more of those symptoms.

In some embodiments, the compound(s) or composition(s) of the invention is administered in the early stages when the subject begins to show elevated glucose levels or increased beta-cell dysfunction, but before complete beta-cell failure. The compound(s) or composition(s) of the invention may also be administered when loss of beta-cell mass appears to be reversible.

In some embodiments, the subject is a human patient with type 1 diabetes. As is known, type 1 diabetes occurs when a person's immune system attacks the insulin producing beta-cells in the pancreas and destroys them such that the pancreas then produces little or no insulin. The most common type 1 diabetes symptoms include excessive thirst (polydipsia), frequent urination (polyuria), extreme hunger (polyphagia), extreme fatigue, and weight loss. In some embodiments, the methods, compounds or compositions of the invention are effective in improving, curing and/or alleviating one or more of those symptoms. In some embodiments, the subject as an autoimmune reaction leading to the destruction and/or apoptosis of beta-cells. In some embodiments, ketones are present in the urine of the subject. The compound(s) or composition(s) of the invention may also be administered when there are early signs of inflammation (e.g. cellular immune response, over production of cytokines (e.g. TNF-alpha, IFN-gamma, IL-1, IL-2 and IL-8).In some embodiments, the administration of the compound(s) or composition(s) of the invention can be initiated (a) before a subject who is at risk for an insulin related disorder, shows clinical symptoms of an insulin related disorder; (b) after the subject begins to show signs of an insulin related disorder, e.g., elevated glucose levels or beta-cell failure (as evidenced, e.g., by an increase or decrease of more than 5, 10, 20, or 30% in glucose levels or beta-cell failure compared to a reference value, e.g., a control, e.g., a non-disease state control); (c) when an insulin related disease, e.g., diabetes or another insulin related disorder described herein is diagnosed; (d) before, during or after a treatment for an insulin related disorder, e.g., diabetes, is begun or begins to exert its effects. The period over which the agent is administered (or the period over which clinically effective levels are maintained in the subject) can be long term, e.g., for six months or more or a year or more, or short term, e.g., for less than a year, six months, one month, two weeks or less.

In some embodiments, the compound(s) or composition(s) of the invention is administered before the subject shows clinical symptoms of a pancreatic disorder, but after a determination that the subject is at risk of pancreatic disorder, e.g., the subject is obese, or the subject has a family history of pancreatic disorders (e.g., a parent, sibling or grandparent of the subject has a pancreatic disorder such as diabetes).

In some embodiments, the compound(s) or composition(s) of the invention is administered as a supplemental therapy for a pancreatic disorder, e.g., the agent is administered in addition to administration of insulin.

In some embodiments, the subject exhibits abnormal pancreatic function (e.g., the subject displays abnormal insulin secretion, the subject displays signs of insulin resistance, the subject has hyperinsulinemia or hyperglycemia, etc).

In some embodiments of the invention, the subject is a non-human animal, such as an animal model of a pancreatic disorder, e.g., the NOD Mouse and its related strains, the BB Rat, leptin or leptin receptor mutant rodents, Zucker Diabetic Fatty (ZDF) Rat, Sprague-Dawley rats, Obese Spontaneously Hypertensive Rat (SHROB, Koletsky Rat), Wistar Fatty Rat, New Zealand Obese Mouse, NSY Mouse, Goto-Kakizaki Rat, OLETF Rat, JCR:LA-cp Rat, Neonatally Streptozotocin-Induced (n-STZ) Diabetic Rats, Rhesus Monkey, Psammomys obesus (fat sand rat), C57B1/6J Mouse, ob/ob mouse, and diabetic Tori rat. In a preferred embodiment, the subject is a mammal, e.g., a human. More preferably, the subject is a human at risk for or having a pancreatic disorder (e.g., type 1 or type 2 diabetes).

Another aspect of the invention relates to a method of treating, preventing or delaying the onset of a condition selected from hyperglycemia, low glucose tolerance, insulin resistance, obesity, lipid disorders, dyslipidemia, hyperlipidemia, hypertrigylceridemia, hypercholesterolemia, low HDL levels, high LDL levels, fatty liver disease, cachexia, atherosclerosis and its sequalea, vascular restenosis, pancreatitis, abdominal obesity, nephropathy, neuropathy, ulceration of the extremities and other conditions where insulin resistance is a component. In another aspect, the invention relates to a method for delaying the requirement for treating a diabetic patient with insulin.

A further aspect of the invention relates to the treatment, prevention or amelioration of pathological states caused by insulin resistance and/or type 2 diabetes, including microvascular complications, such as nephropathy, neuropathy, cataracts and retinopathy; macrovascular complications, such as atherosclerosis, arteriosclerosis, hypertension, coronary heart disease, cerebrovascular disease and peripheral vascular disease; and related morbidities, such as obesity, premature aging, cataracts and possibly Alzheimer's disease.

In some embodiments the compound is administered to the subject in a pharmaceutical composition further comprising a pharmaceutically acceptable vehicle. In some embodiments, the method includes orally administering the pharmaceutical composition. In some embodiments, the method includes intravenously administering the pharmaceutical composition.

The terms “effective amount” or “therapeutically effective amount” are used interchangeably herein and refer to the amount of a compound which is effective to treat a subject, e.g., treat a subject for a pancreatic disorder (e.g. diabetes) or and/or another condition such as metabolic syndrome. The therapeutically effective amount may vary based on the particular disorder(s) the subject is suffering from, the age, weight, and lifestyle of a particular subject. In addition, the therapeutically effective amount may depend on the subject's blood parameters (e.g. lipid profile, insulin levels, glycemia), the severity of the disease state, organ function, kidney function, pancreatic function or underlying disease or complications.

For example, the therapeutically effective amount of the compound of formula (I) may be between about 100 and 4000 mg daily. The compounds of the invention may be manufactured in tablets, pills, or capsules with dosages of 200 mg, 400 mg, or 800 mg, or 1200 mg or 1800 mg of the compound of the invention. In some embodiments, a therapeutically effective amount may be 400 mg BID, 800 mg BID, 1200 mg, 1600 mg, 2400 mg or 3600 mg BID. BID means twice a day. In some embodiments, a therapeutically effective amount is aimed at obtaining serum levels in human patients corresponding to at least 1, 5, 10, 25, 50, 75, or 100 μg/ml.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). In some embodiments, the subject candidate for preventive treatment is a patient at risk of, a patient whom has been diagnosed with, or whom is progressing towards a vascular or a cardiovascular disease, a pancreatic disorder, diabetes, metabolic syndrome, obesity and the like. Biological and physiological parameters for identifying such patients are provided herein and are also well known by physicians.

The terms “treatment” or “treating” of a subject includes the application or administration of a compound of the invention to a subject (or application or administration of a compound of the invention to a cell or tissue from a subject) with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. For example, quantitative assessment of pancreatic function or dysfunction are well known in the art and examples of assays for the determination of pancreas function/dysfunction are given hereinafter and includes evaluating biological and/or physiological parameters such as islets of Langerhans' size, growth and/or secreting activity, beta-cells' size, growth and/or secreting activity; insulin secretion and circulating blood levels, glucose blood levels, and pancreas biopsy.

Examples of compounds of the invention include the compounds in the following table and pharmaceutically acceptable salts thereof.

| | |
|---|---|
| 1,2-Ethanedisulfonic acid | HO₃SCH₂CH₂SO₃H |
| Sodium 1,2-ethanedisulfonate | NaO₃SCH₂CH₂SO₃Na |
| 1,3-propanedisulfonic acid | HO₃SCH₂CH₂CH₂SO₃H |
| Sodium 1,3-propanedisulfonate | NaO₃SCH₂CH₂CH₂SO₃Na |
| (1,3-propanedisulfonic acid, | |
| disodium salt) | |
| 1,2-Ethanediol bis(hydrogen sulfate) | HO₃SOCH₂CH₂OSO₃H |
| 1,2-Ethanediol disulfate, disodium | NaO₃SOCH₂CH₂OSO₃Na |
| salt | |
| 1,3-Propanediol bis(hydrogen sulfate) | HO₃SOCH₂CH₂CH₂OSO₃H |
| 1,3-Propanediol disulfate, disodium | NaO₃SOCH₂CH₂CH₂OSO₃Na |
| salt | |
| 2-Sulfomethyl-1,4-butanedisulfonic | HO₃SCH₂CH₂CH(CH₂SO₃H)₂ |
| acid | |
| 2-Sulfomethylbutane-1,4-disulfonic | NaO₃SCH₂CH₂CH(CH₂SO₃Na)₂ |
| acid, trisodium salt | |

The term “compound” includes chemical entities. The compounds may be in solid, liquid or gaseous phase. The term compound includes the compounds of formula (I) and pharmaceutically acceptable salts thereof. Compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain a chiral center and, therefore, may exist as stereoisomers. Compounds, as defined herein, may be purified from natural sources, purchased from commercial sources or chemically synthesized using art recognized techniques.

In general, all compounds of the present invention may be prepared by any conventional methods, using readily available and/or conventionally preparable starting materials, reagents and conventional synthesis procedures. More particularly, 1,3-propanedisulfonic acid or a pharmaceutically acceptable salt thereof may be prepared by the methods described in U.S. Pat. No. 5,643,562. In addition, the compounds of the invention also may exist in hydrated and anhydrous forms. Hydrates of the compound of formula (I) are included as compounds of formula (I). In a further embodiment, the compound of formula (I) is a monohydrate. In one embodiment, the compound of formula (I) comprises about 10% or less, about 9% or less, about 8% or less, about 7% or less, about 6% or less, about 5% or less, about 4% or less, about 3% or less, about 2% or less, about 1% or less, about 0.5% or less, about 0.1% or less by weight of water. In another embodiment, the compounds of the invention comprise, about 0.1% or more, about 0.5% or more, about 1% or more, about 2% or more, about 3% or more, about 4% or more, about 5% or more, or about 6% or more by weight of water.

In addition, the compounds of the invention may also encompass more than one polymorphic forms, hydrated states, etc. For example, one form, Form I, can be prepared by direct recrystallization of a compound of the invention, e.g., 1,3-propanedisulfonic acid, disodium salt. The compound is precipitated from solution with 16:1 ethanol:water (v/v). The recrystallized product is recovered as a fine white powder which is then dried at 65° C. for 16 hours at 4 mm Hg. The resulting non-hydrated form has a moisture content of 0.2% and an apparent density of 0.64 g/ml. In a further embodiment, the compound of formula (I) has a moisture content of about 0.2%.

Furthermore, another form, Form II, can be prepared by direct recrystallization of a commercially available 1,3-propanedisulfonic acid, disodium salt in a fashion similar to Form I. The compound is precipitated from solution with 8:1 ethanol:water (v/v). The recrystallized product is recovered as a white solid which is then dried at 20-25° C. for 16 hours at 4 mm Hg. The resulting mono-hydrated form has a moisture content of about 7% w/w and an apparent density of 0.46 g/ml. In a further embodiment, the compound of formula (I) has a moisture content of about 7%.

Form I can be also be prepared from the Form II polymorph by prolonged heating at reduced pressures. First, the Form II polymorph (water content 6.8%) is dried at 65° C. for 16 hours in a vacuum at 4 mm Hg. This initial drying reduces the water content of the formerly hydrated polymorph to 2.3%. After another 24 hours at 65° C., the moisture content of the formerly monohydrated polymorph is reduced to 1%. The compound is entirely converted to Form I polymorph only after an additional 48 hours of drying at 77° C.

The compounds of the present invention contain one or more acidic functional groups and, thus, are capable of forming pharmaceutically acceptable salts with pharmaceutically acceptable bases. A “pharmaceutically acceptable salt” of a compound means a salt of a compound that is pharmaceutically acceptable. Desirable are salts of a parent compound that retain or improve the biological effectiveness and properties of the free acids and bases of the parent compound as defined herein, or that takes advantage of an intrinsically basic, acidic or charged functionality on the molecule and that is not biologically or otherwise undesirable. Example of pharmaceutically acceptable salts are also described, for example, in Berge et al., “Pharmaceutical Salts”, J. Pharm. Sci. 66, 1-19 (1977). Such salts include base addition salts, formed when an acidic proton present in the parent compound either is replaced by a metal ion, including, an alkali metal ion (e.g. lithium, sodium, potassium), an alkaline earth ion (e.g. magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, trialkylamine (e.g. with a C₁-C₄ alkyl), tromethamine, N-methylglucamine, piperazine, chloroprocain, procain, choline, lysine and the like.

Pharmaceutically acceptable salts may be synthesized from the parent agent that contains an acidic moiety, by conventional chemical methods. Generally, such salts are prepared by reacting the free acid forms of these agents with a stoichiometric amount of the appropriate base in water or in an organic solvent, or in a mixture of the two. Salts may be prepared in situ, during the final isolation or purification of the agent or by separately reacting a purified compound of the invention in its free acid form with the desired corresponding base, and isolating the salt thus formed.

All acid, salt and other ionic and non-ionic forms of the compounds described are included as compounds of the invention. For example, if a compound is shown as an acid herein, the salt forms of the compound are also included. Likewise, if a compound is shown as a salt and the acid forms are also included.

In a further embodiment, the compound of formula (I) is not 1,3-propanedisulfonic acid disodium salt or 1,3-propanedisulfonic acid.

In a further embodiment, compounds of the invention include compounds disclosed in WO 94/22437, WO 96/28187, and WO 00/64420, the contents of which are hereby incorporated by reference in their entirety.

In a further embodiment, the composition or formulation is not as described in Example 1 or as described in any of the examples. In another further embodiment, at least one ingredient is not an ingredient described in Example 1 or as described in any of the examples.

### Pharmaceutical Compositions

A related aspect of the invention concerns pharmaceutical compositions for use: (i) in preventing or treating renal disorders and more particularly nephropathy, (ii) in preventing or treating renal disorder complications and/or (iii) prevention and/or treatment of dyslipidemias.

A related aspect of the invention concerns the use of a compound of Formula (I) as described herein, preferably 1,3-propanedisulfonic acid or a pharmaceutically acceptable salt thereof, and more preferably 1,3-propanedisulfonic acid sodium salt, in the manufacture of a medicament for use: (i) in preventing or treating a renal disorder and more particularly nephropathy, (ii) in preventing or treating renal disorder complications and/or (iii) prevention and/or treatment of dyslipidemias. As use herein, the terms “pharmaceutical composition” and “medicament” are used interchangeably.

In another preferred embodiment, there is also provided a pharmaceutical composition useful in the prevention and/or treatment of type 1 diabetes, type 2 diabetes, LADA, and/or gestational diabetes, which comprises a therapeutically-effective amount of a compound of Formula (I) as defined herein.

In some embodiments, the compositions of the invention comprise an effective amount of a compound of the Formula (I) as described hereinbefore, preferably 1,3-propanedisulfonic acid or a pharmaceutically acceptable salt thereof, and more preferably 1,3-propanedisulfonic acid sodium salt.

Accordingly, in another embodiment, the present invention relates to pharmaceutical compositions comprising effective amounts of one or more compounds according to Formula (I) herein and a pharmaceutically acceptable vehicle, as well as methods of using and manufacturing such pharmaceutical compositions.

As used herein, the term “pharmaceutical composition” refers to at least one compound and at least one pharmaceutically acceptable vehicle, with which the compound is administered to a subject.

“Pharmaceutically acceptable vehicle” refers to a diluent, adjuvant, excipient, or carrier with which a compound is administered. The term “pharmaceutically acceptable” refers to drugs, medicaments, inert ingredients etc., which the term describes, suitable for use in contact with the tissues of humans and lower animals without undue toxicity, incompatibility, instability, irritation, allergic response, and the like, commensurate with a reasonable benefit/risk ratio. It preferably refers to a compound or composition that is approved or approvable by a regulatory agency of the Federal or state government or listed in the U.S. Pharmacopoeia or other generally recognized pharmacopoeia for use in animals and more particularly in humans.

As used herein, the term “therapeutically effective amount” means the amount of compound that, when administered to a subject for treating or preventing a disease, is sufficient to effect such treatment or prevention of the disease. As indicated hereinbefore, the “therapeutically effective amount” will vary depending on the compound, the disease and its severity, and the age, weight, etc., of the subject in need of treatment.

The compounds of the invention may be formulated prior to administration into pharmaceutical compositions using available techniques and procedures (e.g. US patent application No. US 2006/0252829, which is incorporated herein by reference). For instance, the pharmaceutical compositions are formulated into suitable administration (orally, parenterally, (IV, IM, depo-IM, SC, and depo SC), sublingually, intranasally (inhalation), intrathecally, topically, or rectally). Suitable pharmaceutically acceptable vehicles include, without limitation, any non-immunogenic pharmaceutical carrier or diluent suitable for oral, parenteral, nasal, mucosal, transdermal, topical, intrathecal, rectal, intravascular (IV), intraarterial (IA), intramuscular (IM), and subcutaneous (SC) administration routes, such as phosphate buffer saline (PBS). Also, the present invention includes such compounds which have been lyophilized and which may be reconstituted to form pharmaceutically acceptable formulations for administration, as by intravenous, intramuscular, or subcutaneous injection. Administration may also be intradermal or transdermal.

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Preferably, the compound(s) of the invention can be orally administered. Formulations of the present invention include those suitable for oral administration. The formulations may conveniently be presented in unit dosage form and may be prepared by any methods well known in the art of pharmacy. Methods of preparing these formulations or compositions include the step of bringing into association a compound of the present invention with a pharmaceutically acceptable vehicle (e.g. an inert diluent or an assimilable edible carrier) and, optionally, one or more accessory ingredients. In general, the formulations are prepared by uniformly and intimately bringing into association a compound of the present invention with liquid carriers, or finely divided solid carriers, or both, and then, if necessary, shaping the product. The amount of the therapeutic agent in such therapeutically useful compositions is such that a suitable dosage will be obtained.

Formulations of the invention suitable for oral administration may be in the form of capsules (e.g. hard or soft shell gelatin capsule), cachets, pills, tablets, lozenges, powders, granules, pellets, dragees, e.g., coated (e.g., enteric coated) or uncoated, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of a compound of the present invention as an active ingredient. A compound of the present invention may also be administered as a bolus, electuary or paste, or incorporated directly into the subject's diet. Moreover, in certain embodiments these pellets can be formulated to (a) provide for instant or rapid drug release (i.e., have no coating on them); (b) be coated, e.g., to provide for sustained drug release over time; or (c) be coated with an enteric coating for better gastrointestinal tolerability.

In solid dosage forms of the invention for oral administration the active ingredient is mixed with one or more pharmaceutically acceptable carriers, such as sodium citrate or dicalcium phosphate, or any of the following: fillers or extenders, such as starches, lactose, sucrose, glucose, mannitol, or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Peroral compositions typically include liquid solutions, emulsions, suspensions, and the like. The pharmaceutically acceptable vehicles suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

Pharmaceutical formulations are also provided which are suitable for administration as an aerosol, by inhalation. These formulations comprise a solution or suspension of the desired compound of any Formula herein or a plurality of solid particles of such compound(s). The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid agent of any Formula described herein, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol may be in the form of a liquid, the formulation will comprise a water-soluble agent of any Formula described herein, or a salt thereof, in a carrier which comprises water. A surfactant may be present which lowers the surface tension of the formulation sufficiently to result in the formation of droplets within the desired size range when subjected to nebulization.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or even from about 1% to about 5%, of a compound of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included. The compound(s) of the invention may also be administered parenterally, intraperitoneally, intraspinally, or intracerebrally. For such compositions, the compound(s) of the invention can be prepared in glycerol, liquid polyethylene glycols, and mixtures thereof and in oils. Under ordinary conditions of storage and use, these preparations may contain a preservative to prevent the growth of microorganisms.

To administer the compound(s) of the invention by other than parenteral administration, it may be useful to coat the compound(s) with, or co-administer the compound(s) with a material to prevent its inactivation. For example, the compound(s) of the invention may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes.

Pharmaceutical compositions according to the invention may also be coated by conventional methods, typically with pH or time-dependent coatings, such that the compound(s) of the invention is released in the vicinity of the desired location, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

### Dosage

It is understood that appropriate doses depend upon a number of factors within the knowledge of the ordinarily skilled physician, veterinarian, or researcher (e.g. see Wells et al. eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000)). The dose(s) of the compound(s) of the invention will vary, for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g. bioavailability, stability, potency, toxicity, etc). Such appropriate doses may be determined using any available assays including the assays described herein. When one or more of the compounds of the invention is to be administered to humans, a physician may for example, prescribe a relatively low dose at first, subsequently increasing the dose until an appropriate response is obtained.

For example, the therapeutically effective amount of the compound of Formula (I) may be between about 100 and 4000 mg daily. The compounds of the invention may be manufactured in tablets, pills, or capsules with dosages of 200 mg, 400 mg, or 800 mg, or 1200 mg, or 1800 mg, or 2400 mg of the compound of the invention. In some embodiments, a therapeutically effective amount may be 400 mg BID, 800 mg BID, 1200 mg, 1600 mg, 2400 mg or 3600 mg BID. BID means twice a day. In some embodiments, a therapeutically effective is aimed at obtaining serum levels in human patients corresponding to at least 1, 5, 10, 25, 50, 75, or 100 μg/ml.

Exemplary doses include milligram or microgram amounts of the compound per kilogram of subject or sample weight (e.g., about 1 milligram per kilogram to about 200 milligrams per kilogram, about 5 milligram per kilogram to about 100 milligram per kilogram, about 10 milligram per kilogram to about 50 milligrams per kilogram). Additional exemplary doses include doses of about 1 to about 500 mg, or about 5 to about 300 mg, or about 10 to about 200 mg daily, twice or trice daily, or lower or higher amounts. For comparison, exemplary doses for Eprodisate (1,3-propanedisulfonic acid sodium salt) for the treatment of AA amyloidosis is about 400 mg, 800 mg or 1200 mg BID (two times per day) base on the patient's creatine clearance. See also published US patent application No. US 2006/0252829, which is incorporated herein by reference.

It is generally advantageous to formulate parenteral compositions in dosage unit form for ease of administration and uniformity of dosage. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical vehicle. In an embodiment, the compositions according to the invention are formulated in a unit dosage form, each dosage containing from about 100 mg to about 2000 mg, more preferably about 200 mg to about 1000 mg, even more preferably about 400 mg to about 800 mg of the compound according to the invention. See also published US patent application No. US 2006/0252829, which is incorporated herein by reference. The specification for the dosage unit forms of the invention may vary and are dictated by and directly dependent on (a) the unique characteristics of the therapeutic agent and the particular therapeutic effect to be achieved, and (b) the limitations inherent in the art of compounding such a therapeutic agent for the treatment of amyloid deposition in subjects.

Administration of the compounds and compositions of the present invention to a subject to be treated can be carried out using known procedures, at dosages and for periods of time effective to achieved a desired purposes (e.g. prevention or treatment of nephropathy, improvement of kidney function in general, and/or prevention and/or treatment of a blood lipids-associated condition, etc). Dosage regimens can be adjusted to provide the optimum therapeutic response. For example, several divided doses may be administered daily or the dose may be proportionally reduced as indicated by the exigencies of the therapeutic situation.

In one embodiment, the compound(s) of the invention is administered at a therapeutically effective dosage sufficient to positively affect, impact and/or modify a kidney function parameter such as albuminuria, proteinuria, creatinine clearance, urea clearance. In another embodiment, the compound(s) of the invention is administered at a therapeutically effective dosage sufficient to positively affect, impact and/or modify circulating blood levels and/or ratios of triglycerides, cholesterol, high-density lipoprotein cholesterol (HDLC), very low density lipoprotein cholesterol (VLDLC), low density lipoprotein cholesterol (LDLC), intermediate density lipoprotein cholesterol, low density lipoprotein (LDL), high density lipoprotein (HDL), and free fatty acids.

When referring to a positive effect, impact and/or modification of a kidney function parameter or circulating blood levels a “therapeutically effective” dosage refers to a modification (e.g. slowing of decline of renal function, lowering circulating harmful lipids levels) for example, of at least about 1%, or by at least about 5%, or by at least about 10%, or by at least about 20%, or by at least about 40%, or by at least about 50%, or by at least 60%, or by at least 75%, or even by at least about 100%,or more relative to untreated subjects.

### Co-Administration

The method of treatment of the present invention may also include co-administration of the at least one compound according to the invention, e.g., 1,3-propanedisulfonic acid or a pharmaceutically acceptable salt thereof together with the administration of another therapeutically effective agent for the prevention or treatment of a renal disorder or complication, nephropathy (e.g. diabetic nephropathy), diabetes, dyslipidemia, hypertension and/or obesity.

In one embodiment, such co-administration of a compound of the invention with a second agent may allow lowering of the necessary dosage of the second agent such that co-administration, for examples, decreases side effects, or improves blood glucose levels control. Co-administration may also prevent, treat or lessen one or more symptoms or features of metabolic syndrome, or reduce the risk of diabetes-related health complications.

In one embodiment, the compound(s) of the invention is used in combination with at least one additional known compound which is currently being used or is in development for preventing or treating diabetes. Examples of such known compounds include but are not limited to common anti-diabetic drugs such as sulphonylureas (e.g. glicazide, glipizide), metformin, glitazones (e.g. rosiglitazone, pioglitazone), prandial glucose releasing agents (e.g. repaglinide, nateglinide) and acarbose. A more detailed but non-limitative list of useful antidiabetic compounds or agents that can be used in combination with the compound(s) of the invention include insulin, biguanides, such as, for example metformin (Glucophage®, Bristol-Myers Squibb Company, U.S.; Stagid®, Lipha Sante, Europe); sulfonylurea drugs, such as, for example, gliclazide (Diamicron®), glibenclamide, gilpizide (Glucotrot® and Glucotrol XL®, Pfizer), glimepiride (Amaryl®, Aventis), chlorpropamide (e.g., Diabinese®, Pfizer), tolbutamide, and glyburide (e.g., Micronase®, Glynase®, and Diabeta®); glinides, such as, for example, repaglinide (Prandin® or NovoNorm®; Novo Nordisk), ormitiglinide, nateglinide (Starlix®), senaglinide, and BTS-67582; DPP-IV inhibitors such as vildagliptin and sitagliptin; insulin sensitizing agents, such as, for example, glitazones, a thiazolidinedione such as rosiglitazone maleate (Avandia®, Glaxo SmithKline), pioglitazone (Actos®, Eli Lilly, Takeda), troglitazone, ciglitazone, isaglitazone, darglitazone, englitazone; glucagon-like peptide I (GLP-1) receptor agonists, such as, for example, Exendin-4 (1-39) (Ex-4), Byetta™ (Amylin Pharmaceuticals Inc.), CJC-1 131 (Conjuchem Inc.), NN-221 I (Scios Inc.), GLP-1 agonists as those described in WO 98/08871; agents that slow down carbohydrate absorption, such as, for example, a-glucosidase inhibitors (e.g., acarbose, miglitol, voglibose, and emiglltate); agents that inhibit gastric emptying, such as, for example, glucagon-like peptide 1, cholescystokinin, amylin, and pramlintide; glucagon antagonists, such as, for example, quinoxaline derivatives (e.g., 2-styryl-3-[3-(dimethylamino)propylmethylamino1-6, 7-dichloroquinoxaline, Collins et al., Bioorganic and Medicinal Chemistiy Letters 2(9):91 5-91 8, 1992), skyrin and skyrin analogs (e.g., those described in WO 94/14426), 1-phenyl pyrazole derivatives (e.g., those described in U.S. Pat. No. 4,359,474), substituted disllacyclohexanes (e.g., those described in U.S. Pat. No. 4,374,130), substituted pyridines and biphenyls (e.g., those described in WO 98/04528), substituted pyridyl pyrroles (e.g., those described in U.S. Pat. No. 5,776,954), 2,4-diaryl-5-pyridylimidazoles (e.g., those described in WO 98/21957, WO 98/22108, WO 98/22109, and U.S. Pat. No. 5,880,139), 2,5-substituted aryl pyrroles (e.g., those described in WO 97/1 6442 and U.S. Pat. No. 5,837,719), substituted pyrimidinone, pyridone, and pyrimidine compounds (e.g., those described in WO 98/24780, WO 98/24782, WO 99/24404, and WO 99/32448), 2-(benzirnidazol-2-ylthio)-1-(3,4-dihydroxyphenyl)-1-ethanones (see Madsen et aL, J. Med. Chem. 41:5151-5157, 1998), alkylidene hydrazides (e.g., those described in WO 99/01423 and WO 00/39088), glucokinase activators, such as, for example, those described in WO 00/58293, WO 01/44216, WO 01/83465, WO 01/83478, WO 01/85706, and WO 01/85707 and other compounds, such as selective ADP-sensitive K⁺ channels activators (e.g. diazoxide), hormones (e.g. cholecytokinin, GRP-bombesin, and gastrin plus EGF receptor ligands; see Banerjee et al. Rev Diabet Stud, 2005 2(3): 165-176); peroxisome proliferator-activated receptor-gamma (PPAR-gamma) agonist (e.g. pioglitazone; see Ishida et al., Metabolism, 2004, 53(4), 488-94); antioxydants (e.g. 1-bis-o-hydroxycinnamoylmethane, curcuminoid bis-demethoxycurcumin; see Srivivasan et al., J Pharm Pharm Sci. 2003, 6(3): 327-33), WO 00/69810, WO 02/00612, WO 02/40444, WO 02/40445, WO 02140446, and the compounds described in WO 97/41097 (DRF-2344), WO 97/41119, WO 97/41120, WO 98/45292, WO 99/19313 (NN622/DRF-2725), WO 00/23415, WO 00/23416, WO 00/23417, WO 00/23425, WO 00/23445, WO 00/23451, WO 00/41121, WO 00/50414, WO 00/63153, WO 00/63189, WO 00/63190, WO 00/63191, WO 00/63192, WO 00/63193, WO 00/63196, WO 00/63209, U.S. Pat. No. 6,967,019, U.S. Pat. No. 7,101,845, U.S. Pat. No. 7,074,433, U.S. Pat. No. 6,992,060, U.S. Pat. No. RE39,062, WO 2006/131836 and WO 2006/120574; and the compounds referred to in the public domain as T-174, GI-262570, YM-440, MCC-555, JTT-501, AR-H039242, KRP-297, GW-409544, CRE-16336, AR-H049020, LY510929, MBX-102, CLX-0940, and GW-501516.

Additional examples of agents that can be co-administered with the compound(s) according to the invention are compounds for stimulating pancreatic beta-cell neogenesis and/or regeneration of islets. Examples of compounds currently used or in development which have a positive effect on islet number (i.e. beta-cells) include Byetta™ (exendin-4 inhibitor), vildagliptin (Galvus™, dipeptidylpeptidase inhibitor), Januvia™ (sitagliptin phosphate) and extracts from Gymnema sylvestrae leaf (Pharma Terra). The compound(s) according to the invention may also be administered with biomolecules related to cell regeneration such as β-cellulin, plant extracts from Beta vulgaris or Ephedra herba, and nicotinamide (see Banerjee et al. Rev Diabet Stud, 2005 2(3): 165-176).

Additional compounds or agents that may be used in accordance with the principles of the present invention are those capable of inducing pancreatic beta-cell growth or insulin producing cell growth and/or insulin production. Such compounds include, but are not limited to: glucagon-like peptide-1 (GLP-1) and long-acting, DPP-IV-resistant GLP-1 analogs thereof, GLP-1 receptor agonists, gastric inhibitory polypeptide (GIP) and analogs thereof (e.g., which are disclosed in U.S. Patent Publication No. 20050233969), dipeptidyl peptidase IV (DPP-IV) inhibitors, insulin preparations, insulin derivatives, insulin-like agonists, insulin secretagogues, insulin sensitizers, biguanides, gluconeogenesis inhibitors, sugar absorption inhibitors, renal glucose re-uptake inhibitors, β3 adrenergic receptor agonists, aldose reductase inhibitors, advanced glycation end products production inhibitors, glycogen synthase kinase-3 inhibitors, glycogen phosphorylase inhibitors, antilipemic agents, anorexic agents, lipase inhibitors, antihypertensive agents, peripheral circulation improving agents, antioxidants, diabetic neuropathy therapeutic agents, and the like.

In one embodiment, the compound(s) of the invention is used in combination with at least one additional known compound which is currently being used or in development for preventing or treating renal disorder such as nephropathy, or an associated disorder or complication. Examples of such known compounds include but are not limited to: ACE inhibitor drugs (e.g. captopril (Capoten®), enalapril (Innovace®), fosinopril (Staril®), lisinopril (Zestril®), perindopril (Coversyl®), quinapril (Accupro®), trandanalopril (Gopten®), lotensin, moexipril, ramipril); RAS blockers; angiotensin receptor blockers (ARBs) (e.g. Olmesartan, Irbesartan, Losartan, Valsartan, candesartan, eprosartan, telmisartan, etc); protein kinase C (PKC) inhibitors (e.g. ruboxistaurin); inhibitors of AGE-dependent pathways (e.g. aminoguanidine, ALT-946, pyrodoxamine (pyrododorin), OPB-9295, alagebrium); anti-inflammatory agents (e.g. clyclooxigenase-2 inhibitors, mycophenolate mophetil, mizoribine, pentoxifylline), GAGs (e.g. sulodexide (U.S. Pat. No. 5,496,807)); pyridoxamine (U.S. Pat. No. 7,030,146); endothelin antagonists (e.g. SPP 301), COX-2 inhibitors, PPAR-γ antagonists and other compounds like amifostine (used for cisplatin nephropathy), captopril (used for diabetic nephropathy), cyclophosphamide (used for idiopathic membranous nephropathy), sodium thiosulfate (used for cisplatin nephropathy), tranilast, etc. (Williams and Tuttle (2005), Advances in Chronic Kidney Disease, 12 (2):212-222; Giunti et al. (2006), Minerva Medica, 97:241-62).

Additionally, the methods of the invention may also include co-administration of at least one other therapeutic agent for the treatment of another disease directly or indirectly related to diabetes and/or renal disorder complications, including but not limited to: dyslipidemia, hypertension, obesity, neuropathy, inflammation, and/or retinopathy, etc. Additional examples of agents that can be co-administered with the compound(s) according to the invention are corticosteroids; immunosuppressive medications; antibiotics; antihypertensive and diuretic medications (such as thiazide diuretics and ACE-inhibitors or β-adrenergic antagonists); lipid lowering agents such as bile sequestrant resins, cholestyramine, colestipol, nicotinic acid, and more particularly drugs and medications used to reduce cholesterol and triglycerides (e.g. fibrates (e.g. Gemfibrozil®) and HMG-CoA inhibitors such as Lovastatin®, Atorvastatin®, Fluvastatin®, Lescol®, Lipitor®, Mevacor®, Pravachol®, Pravastatin®, Simvastatin®, Zocor®, Cerivastatin®, etc); compounds that inhibit intestinal absorption of lipids (e.g. ezetiminde); nicotinic acid; and Vitamin D.

Additional examples of agents that can be co-administered with the compound(s) according to the invention are immunomodulating agents or immuno suppressants such as those that are used by type 1 diabetics who have received a pancreas transplant and/or kidney transplant (when they have developed diabetic nephropathy) (see Vinik Al et al. Advances in diabetes for the millennium: toward a cure for diabetes. Med Gen Med 2004, 6:12).

Additional examples of agents that can be co-administered with the compound(s) according to the invention are anti-obesity agents, and appetite reducers. Examples of anti-obesity agents that can be used with the compounds according to the invention include Xenical™ (Roche), Meridia™ (Abbott), Acomplia™ (Sanofi-Aventis), and sympathomimetic phentermine. A non-limitative list of potentially useful known and emerging anti-obesity agents is set forth in Table 2 of WO 2006/131836, that table being incorporated herein by reference.

The compound(s) according to the invention may also be co-administered with known agents that are used to treat hyperkalemia and/or to reduce the risk of ventricular fibrillation caused by hyperkalemia (e.g. calcium gluconate, insulin, sodium bicarbonate, β₂-selective catacholamine such as salbutamol (albuterol, Ventolin®), and polystyrene sulfonate (Calcium Resonium, Kayexalate)).

Therefore, an additional aspect of the invention relates to methods of concomitant therapeutic treatment of a subject, comprising administering to a subject in need thereof an effective amount of a first agent and a second agent, wherein said agent is as defined in Formula (I), and the second agent is for the prevention or treatment of renal disorders, nephropathies, diabetic nephropathy, diabetes, hypertension, hyperlipidemia, metabolic syndrome or obesity.

The invention also relates to the use of at least one first agent as defined in Formula (I), and at least one second agent selected from compounds for the prevention or treatment of renal disorders, nephropathies, diabetic nephropathy, diabetes, hypertension, hyperlipidemia or obesity, for the manufacture of a medicament or kit of medicaments for the concomitant therapeutic treatment or prophylaxis of renal disorders, nephropathies, diabetic nephropathy, diabetes, hypertension, hyperlipidemia, metabolic syndrome or obesity.

As used herein, the term “concomitant” or “concomitantly” as in the phrases “concomitant therapeutic treatment” or “concomitantly with” includes administering a fist agent in the present of a second agent. A concomitant therapeutic treatment method includes methods in which the first, second, third or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g. a human). Preferably the first agent is 3-propanedisulfonic acid or a pharmaceutically acceptable salt thereof, e.g. a disodium salt. The second agent may be selected from the list of compounds given hereinbefore.

Accordingly, the invention also provides a method for preventing, reducing or eliminating a symptom or complication of any one of the above mentioned disease or condition (e.g. diabetes, nephropathy or complication directly or indirectly related to diabetes). The method comprises administering, to a subject in need thereof, a first pharmaceutical composition comprising at least one compound of the invention and a second pharmaceutical composition comprising one or more additional active ingredients, wherein all active ingredients are administered in an amount sufficient to inhibit, reduce, or eliminate one or more symptoms or complications of the disease or condition to be treated. In one aspect, the administration of the first and second pharmaceutical composition is temporally spaced apart by at least about two minutes.

Furthermore, many of the known hypoglycemic agents exhibit undesirable side effects and are toxic in certain cases. For example, in the case of the diabetic patients with seriously lowered pancreatic insulin secretion, effectiveness of insulin secretagogues and insulin sensitizers is diminished. Similarly, in the case of the diabetic patients whose insulin resistance is significantly high, effectiveness of insulin preparations and insulin secretagogues is diminished. Furthermore, there are serious drawbacks associated with prescription of thiazolidindiones (e.g. rosiglutazone) to diabetic patients, including weight gain, fluid retention and increased risks of heart failure. Accordingly, another aspect, the invention relates to a method of reducing undesirable side effects of hypoglycemic agents, the method comprising administering the compound(s) or composition(s) of the invention, preferably 1,3-propanedisulfonic acid and/or 1,3-propanedisulfonic acid sodium salt, concomitantly with a reduced dosage of an hypoglycemic agent (e.g., insulin), thereby achieving substantially the same therapeutic efficacy (e.g. reduction of glycemia to a desired level) when compared to an administration of a higher dosage of the hypoglycemic agent, in absence of the compound(s) or composition(s) of the invention. In another related aspect, the invention relates to a method of preventing gaining of weight and/or fluid retention of thiazolidindiones (e.g., rosiglitazone), the method comprising administering the compound(s) or composition(s) of the invention, preferably 1,3-propanedisulfonic acid and/or 1,3-propanedisulfonic acid sodium salt, concomitantly with a reduced dosage of the thiazolidindione thereby achieving substantially the same therapeutic efficacy and/or reducing risks of heart failure.

### Kits

The compound(s) of the invention may be packaged as part of a kit, optionally including a container (e.g. packaging, a box, a vial, etc). The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The compound(s) of the invention may or may not be administered to a patient at the same time or by the same route of administration. Therefore, the methods of the invention encompass kits which, when used by the medical practitioner, can simplify the administration of appropriate amounts of two or more active ingredients to a patient.

A typical kit of the invention comprises a unit dosage form of a at least one compound according to the invention, e.g., 1,3-propanedisulfonic acid or a pharmaceutically acceptable salt thereof, and a unit dosage form of at least one additional active ingredient. Examples of additional active ingredients that may be used in conjunction with the compounds according to the invention, include, but are not limited to any of the compounds that could be used in combination with the compound(s) of the invention listed herein before in the section “Co-administration”.

Kits of the invention can further comprise devices that are used to administer the active ingredients. Examples of such devices include, but are not limited to, syringes, drip bags, patches, inhalers, enemas, and dispensers for the administration of suppository formulations.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### Assessment of Renal Function, Lipids Profiles and Pancreas Function

In order to evaluate, assess, and/or confirm the efficacy of the method, compounds and/or compositions of the invention, serial measurements can be determined.

Quantitative assessment of renal function and parameters of renal dysfunction are well known in the art and can be found, for example, in Levey (Am J Kidney Dis. 1993, 22(I):207-214). Examples of assays for the determination of renal function/dysfunction are: serum creatinine level; creatinine clearance rate; cystatin C clearance rate, 24-hour urinary creatinine clearance, 24-hour urinary protein secretion; Glomerular filtration rate (GFR); urinary albumin creatinine ratio (ACR); albumin excretion rate (AER); and renal biopsy.

Quantitative assessment of pancreatic function and parameters of pancreatic dysfunction or insufficiency are well known in the art. As mentioned hereinbefore, examples of assays for the determination of pancreas function/dysfunction includes evaluating at least one pancreatic function as assessed using biological and/or physiological parameters such as islets of Langerhans size, growth and/or secreting activity, beta-cells size, growth and/or secreting activity; insulin secretion and circulating blood levels, glucose blood levels, imaging of the pancreas, and pancreas biopsy. For instance, the examples in U.S. Pat. No. 5,424,286 describe methods for testing a compound stimulation of pancreatic insulin secretion, for testing a compound insulinotropic activity or for testing a compound activity on glycemia.

The compounds of the invention may be tested for activity in animal models. Examples of animals models of type II diabetes and obesity include but are not limited to: the Ob/Ob mouse (monogenic model of obesity, leptin deficient), the db/db mouse (monogenic model of obesity, leptin resistant), the Zucker (fa/fa) rat (monogenic model of obesity, leptin resistant), the Goto-Kakizaki rat, the KK mouse, the NSY mouse, the OLETF rat, the Israeli sand rat, the Fat-fed streptozotocin-treated rat, the CBA/Ca mouse, the Diabetic Torri rat, the New Zealand obese mouse (see Rees and Alcolado (2005), Diabet. Med. 22, 359-370), the NOD Mouse and its related strains, the BB Rat, leptin or leptin receptor mutant rodents, and Obese Spontaneously Hypertensive Rat (SHROB, Koletsky Rat).

Known animal models of spontaneous type 2 diabetic nephropathy include: the spontaneously hypertensive/NIH-corpulent (SHR/N-cp) rat (model of obesity, type 2 diabetes and nephropathy), the lean SHR/N-cp rat and the Wistar-Kyoto/NIH-corpulent (WKY/N-cp) rat (both allow assessment of the role of hypertension and obesity in the pathogenesis of diabetic nephropathy: the SHR/N-cp rats have abnormal glucose tolerance, hypertension, and develop a renal disease reminiscent of human diabetic nephropathy, whereas the WKY/N-cp rats are also obese and have hyperlipidaemia, but their glucose control is somewhat worse than that of the SHR/N-cp rat), and the LA/N-cp rat (also carries the gene for obesity, and exhibits hyperlipidaemia) (see Kimmel et al. (1992), Acta Diabetologica, Volume 29 (3-4), 142-148.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The contents of all references, issued patents, and published patent applications cited throughout this application are hereby incorporated by reference. The invention is further illustrated by the following examples, which should not be construed as further limiting.

### EXAMPLES

The Examples set forth herein below provide exemplary formulations of certain representative compounds of the invention. Also provided are exemplary methods for assaying the compounds of the invention for use in the prevention and treatment of diabetes, metabolic syndrome, renal damage and related complications.

Unless otherwise indicated, all numbers expressing quantities of ingredients, reaction conditions, concentrations, properties, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

### Example 1

An example of a formulation of a 400 mg capsule of 1,3 propanedisulfonic acid disodium salt is described below.

Capsules of 400 mg of 1,3 propanedisulfonic acid disodium salt were manufactured by filling # 0 white opaque hard gelatin capsules with a white powder comprised of 400 mg of 1,3 propanedisulfonic acid disodium salt and 40 mg of excipients.

| | | | Label | |
|---|---|---|---|---|
| Raw Material | Grade | Function | (mg/unit) | % |
| 1,3 Propanedisulfonic Acid | MHS* | active | 400.0 | 90.9 |
| Disodium Salt (PDS) | | | | |
| Lactose Monohydrate | NF | diluent | 37.8 | 8.6 |
| (316 Fast-Flo) | | | | |
| Magnesium Stearate | NF | lubricant | 2.2 | 0.5 |
| Sub-Total | | | 440.0 | 100.0 |
| # 0 Hard Gelatin Capsule | MHS* | capsule | 96.0 | |
| Total | | | 536.0 | |

*MHS—Manufacturer House Standard

### Example 2

A pharmaceutical composition is formulated as described in Example 1 with 1,3 propanedisulfonic acid as the active agent.

### Example 3

A pharmaceutical composition is formulated as described in Example 1 with 1,2-ethanedisulfonic acid as the active agent.

### Example 4

A pharmaceutical composition is formulated as described in Example 1 with sodium 1,2-ethanedisulfonate as the active agent.

### Example 5

A pharmaceutical composition is formulated as described in Example 1 with 1,2-ethanediol bis(hydrogen sulfate) as the active agent.

### Example 6

A pharmaceutical composition is formulated as described in Example 1 with 1,2-ethanediol disulfate disodium salt as the active agent.

### Example 7

A pharmaceutical composition is formulated as described in Example 1 with 1,3-propanediol bis(hydrogen sulfate) as the active agent.

### Example 8

A pharmaceutical composition is formulated as described in Example 1 with 1,3-propanediol disulfate disodium salt as the active agent.

### Example 9

A pharmaceutical composition is formulated as described in Example 1 with 2-sulfomethyl-1,4-butanedisulfonic acid as the active agent.

### Example 10

A pharmaceutical composition is formulated as described in Example 1 with 2-sulfomethylbutane-1,4-disulfonic acid trisodium salt as the active agent.

### Example 11

### In vivo Preventive Study of Renal Function, Metabolic Status and Pancreatic Function

The compound 1,3 Propanedisulfonic Acid Disodium Salt (PDS) was selected for a preventive study of renal function in the Zucker rat (ZDF) model as well as its effect on metabolic status and diabetes.

A leading study model for DN is the inbred Zucker Diabetic Fatty rat (ZDF). Given a diabetogenic diet, the ZDF rat will closely mimic human adult onset diabetes (Type 2) and related complications including glomerulosclerosis and renal damage earlier than when fed a normal diet (i.e. 14-18 weeks of age). In addition, obesity, mild hypertension, hypertriglyceridemia, hypercholesterolemia, fasting hyperglycemia, impaired glucose tolerance and hyperinsulinemia, are all major phenotypes featured in the ZDF rat.

### Methods

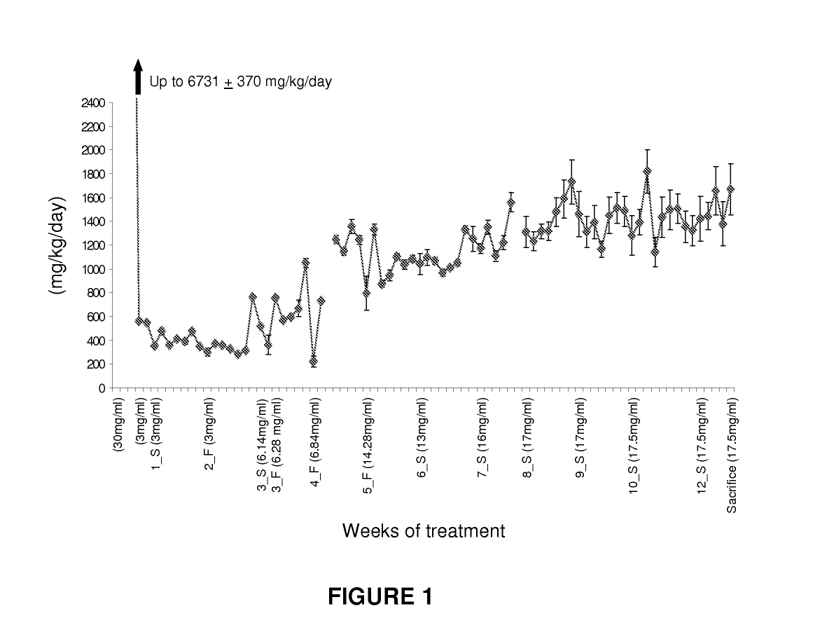

Thirty-two, 6 week-old male ZDF rats (Charles River, St. Constant, Canada) were randomized in 2 groups, Treated (PDS; in 1% sucrose drinking solution) and Control (1% sucrose drinking solution), and studied for a period of 12 weeks. PDS was initially given in high dose (avg: 4270 mg/kg/day) during week 1, followed by an intermediate low dose (avg: 592 mg/kg/day) during weeks 2-5, and finally slightly increased during weeks 6-12 (FIG. 1). All rats were fed a high fat/high sucrose diabetogenic diet (Harlan™ TD95217). Body weight, food and drinking solution consumption were measured on a daily basis. Twelve rats from each group were individually housed in metabolic cages for a period of 24 hours once a week. During week 2, 3, 4 and 5, rats placed in metabolic cages received drinking solution but were placed in fasting condition, whereas during weeks 1, 3, 6 to 12, rats were given ad libitum access to food and drinking solution. At the end of each metabolic cage session, urine output was measured, and blood and urine samples were collected in order to quantify serum and/or urine levels of PDS, creatinine, protein, uric acid, triglycerides, glucose, and electrolytes. These variables were used to calculate creatinine clearance (C_{cr}) and proteinuria, and to evaluate general metabolic and renal health status. At the end of the study, the animals were sacrificed and selected organs were kept for further analysis (e.g. weighing, histology).

a) Renal Function and Metabolism

### Background

Diabetic nephropathy (DN) is the most common cause of chronic kidney failure and end-stage renal disease. Increasing evidence suggests that dyslipidemia, a condition ubiquitously observed in diabetes, is a major independent contributing factor to the progression of DN.

### Aim

This pre-clinical investigation evaluates the role and efficacy of compound 1,3-Propanedisulfonic Acid Disodium Salt (PDS) (Eprodisate Disodium) as a preventive treatment for DN and related pathophysiology in the ZDF rat model. The primary measured outcome is the attenuation/reversal of creatinine clearance deterioration and of proteinuria. The secondary measured outcome is the impact on the metabolic status in this model.

### Results

The results are presented in FIGS. 1 to 7. Results for each time point are represented as median or mean ± SEM. Trend statistics are calculated by two-way ANOVA with or without repeated measures, with p<0.05 considered statistically significant.

Treated animals were given daily an increased amount of PDS as the study progressed (FIG. 1). There was not significant difference in the bodyweight of the treated vs. the control animals (data not shown). As expected, the bodyweight of the animals increased over the study from about 175 g to about 525 g after 12 weeks. A little decline in body weight was observed at the beginning of the study due to diarrhea associated with the higher concentration of PDS.

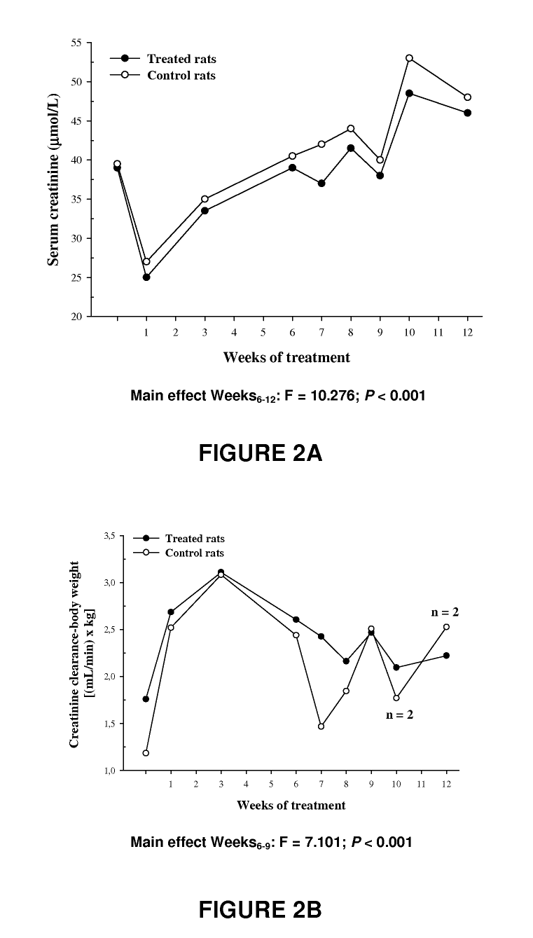

Serum creatinine was stable in both groups throughout the study although there was a tendency for lower values between treatment weeks 6 and 9 in the treated animals, suggesting a greater excretory capacity in these (FIG. 2A). Over the 12 weeks duration of the treatment, there was no significant difference in creatinine clearance despite a slight tendency to be higher between treatment weeks 6 and 8 in the PDS-treated rats (FIG. 2B).

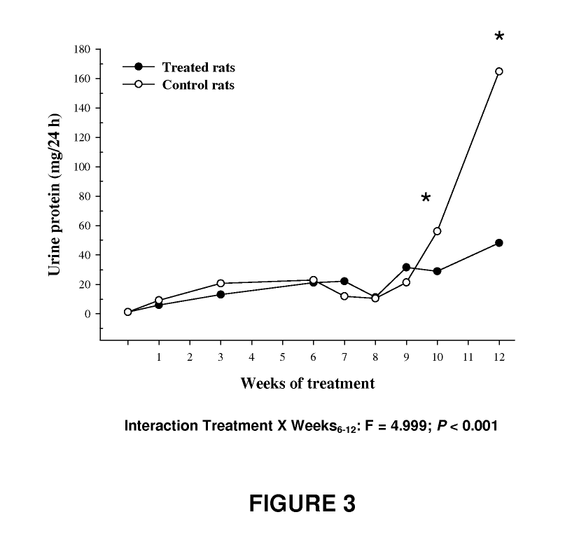

After 8 weeks of treatment, PDS had a measurable effect on proteinuria (FIG. 3), the difference in proteinuria between the control and treated groups was significant at weeks ten and twelve of treatment. The higher proteinuria exhibited by the control group animals may be indicative of more damaged kidneys that are starting to leak plasma macromolecules. The fact that PDS-treated animals show a lesser proteinuria suggests some beneficial effects of the drug on kidney function, especially glomerular integrity.

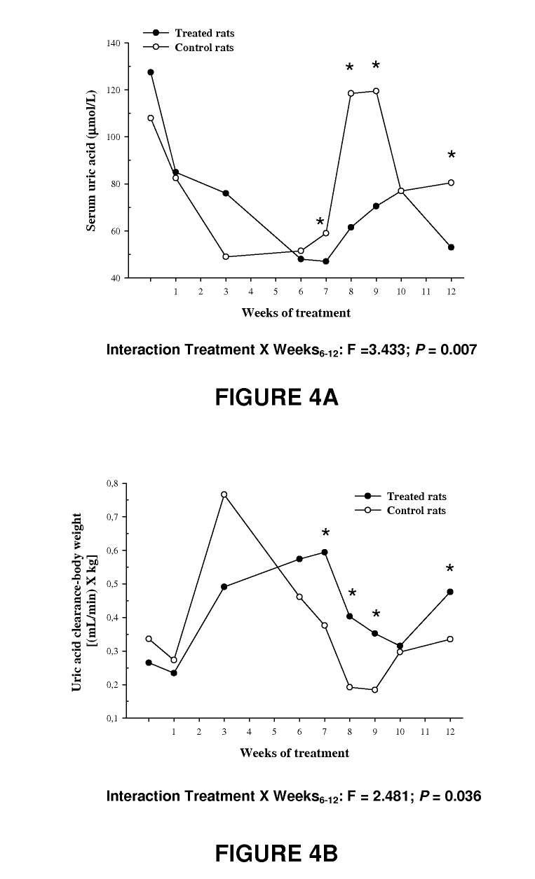

Uric acid is a product of purine metabolism. Animals on the same diet should display similar serum uric acid concentrations. Uric acid levels in serum of animals from the control groups were variable and consistently higher than values from the PDS-treated animals at weeks 6 to 12 (FIG. 4A). Those results suggest a poorer ability to excrete uric acid in control animals. This again may reflect a deficient kidney function in control animals when compared to the PDS-treated rats. As shown in FIG. 4B, clearance of uric acid was also better in treated animals.

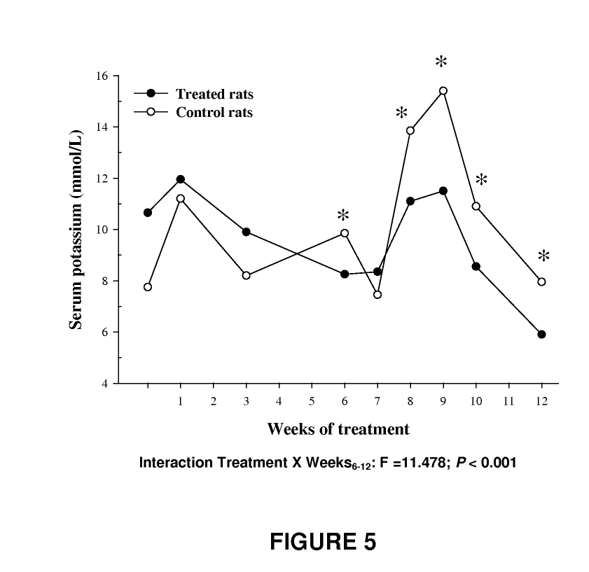

Starting at week 7, both groups show a marked hyperkalemia (i.e. elevated blood level of the electrolyte potassium), significantly more severe in the control group (FIG. 5). Since the animals were fed the same diet, and in absence of any indication suggesting more tubular reabsorption, it is reasonable to conclude that this hyperkalemia was due to an inability of the kidney to excrete potassium. Because it was significantly higher in the control animals (weeks 8 to 12, p<0.001), this suggests that PDS somewhat improved the kidney function of treated animals.

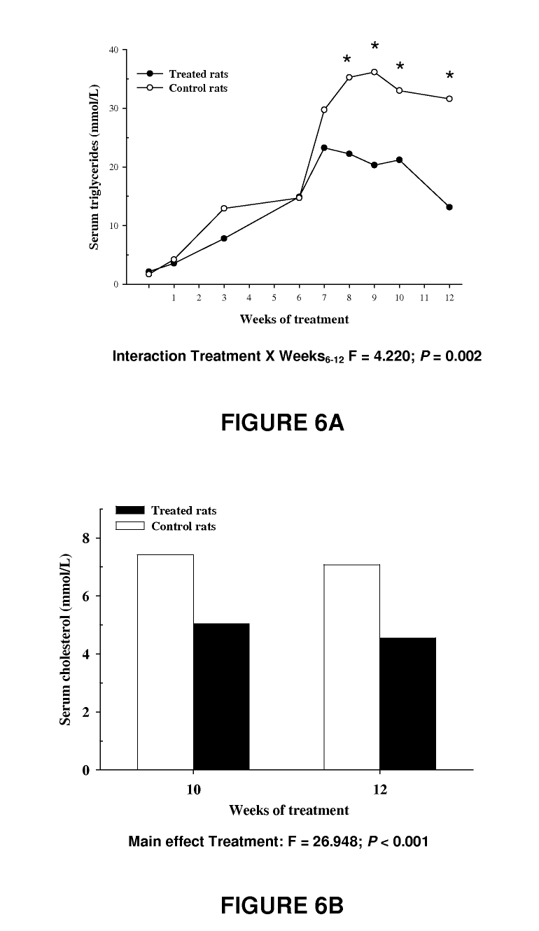

As shown in FIG. 6A, serum triglycerides were consistently lower in PDS-treated rats as compared to controls. This difference was observed from the onset of the treatment, maintained throughout the study and is significant (p=0.002). This strongly suggests a significant impact of PDS on lipid metabolism. Serum cholesterol measured at treatment week 10 (see FIG. 6B) was also significantly lower in the PDS-treated group, a difference that persisted until week 12 (4.5 versus 7.1 mmol/L, p<0.001)).

Organ to body weight (BW) ratios are often used as markers of pathologic states or for an indication of an ongoing remodeling. For instance, in animals as well as in humans, some forms of hypertension are characterized by the hypertrophy of the left cardiac ventricle. This is easily measured by the ratio of heart weight to body weight. Although not shown, no significant difference was observed for heart weight/BW, pancreas weight/BW and kidney weight/BW ratios. Kidneys of the treated animals tended to be smaller than kidneys from the controls. However, a highly significant difference was observed for the ratio of the liver weight/BW (p<0.001) and adrenal weight/body weight (p<0.012) between the groups. This suggests an effect of the PDS treatment on the liver. These phenomena could be related to a lessening of hypertension, although this was not measured in the study. No amyloid deposit was detected in the kidneys of either group.

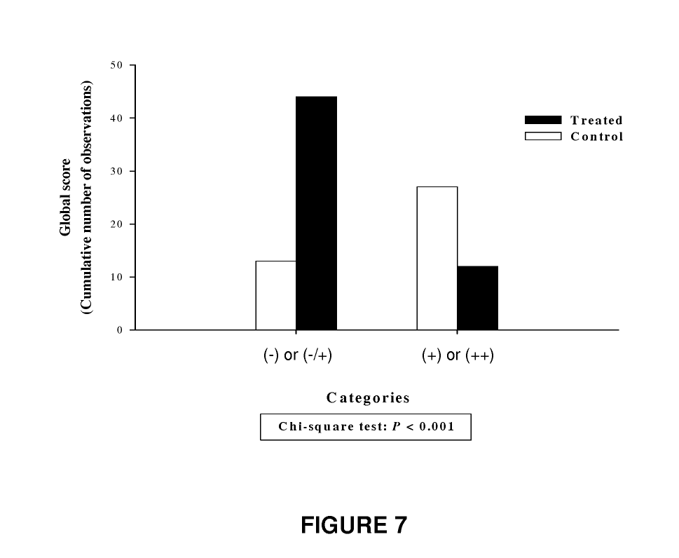

Histology was performed on both controls and PDS-treated animals. As shown in FIG. 7, it was observed from the global score, that most PDs-treated rats had the lowest scores, and the ones from control animals the highest (p=0.001). The global score was calculated by summing up the number of observations for each histological parameter (mesangial matrix expansion, glomerular cell proliferation, basement membrane thickening, and glomerular enlargement). This is a significant indication of the protective ability of PDS on kidney. Although not shown separately, kidneys of the control rats showed glomerular enlargement, glomerular cell proliferation and basement membrane thickening. Kidneys from PDS-treated rat exhibited less pathology. Those results suggest a beneficial effect of PDS on the prevention or treatment of basement membrane alterations (e.g. basement membrane thickening) that are for example, hallmarks of diabetic and/or chronic kidney disease, or other diseases involving the basement membrane. PDS could also be helpful in reversing the subsequent lesions (or scarring or fibrosis, etc.) in these diseases.

Taken together, those results suggest that 1,3 Propanedisulfonic Acid Disodium Salt (PDS) protects the kidneys of Zucker obese diabetic rats. This is demonstrated by the lower proteinuria exhibited in the treated animals as well as by the histology results. There seems to be a general protective effect on the glomeruli that could be attributed to PDS. Results from natriuresis, creatinine clearance and uric acid clearance also suggest a protective effect on the kidney. In addition, PDS may impact lipid metabolism significantly, given the highly significant decreases in triglycerides and cholesterol observed at the end of the study in PDS-treated rats.

b) Effect on the Pancreatic Function

### Background

As is known, diabetes may be due to a lack of insulin secretion (type I diabetes), or to a resistance of peripheral tissues to insulin action (more insulin is needed to decrease blood glucose to the same level as controls in the same amount of time; type II diabetes). The Zucker obese diabetic rat employed in this study is a model of insulin resistance. Hyperglycemia is almost always observed in this model, despite the initially very high circulating insulin levels that fall down with the progression of the disease due to pancreatic beta cell exhaustion. A beneficial treatment is expected to increase insulin secretion, decrease glucose levels by other means or increase glucose utilization by peripheral tissues.

### Results

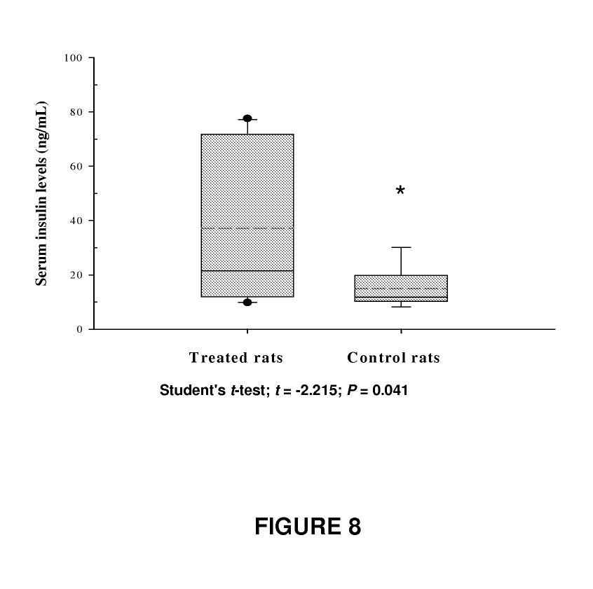

The results are presented in FIGS. 8 to 11. Results for each time point are represented as median and mean ± SEM. Trend statistics are calculated by two-way ANOVA with or without repeated measures, with p<0.05 considered statistically significant.

As shown in FIG. 8, satient insulin was more than twice as high in PDS-treated rats as compared to control animals at the end of the experiment, attesting preservation of beta cell secretory capacity. Because the measurement was performed in fed animals, it suggests an increased insulin secretory capacity in the PDS-treated animals compared to the controls.

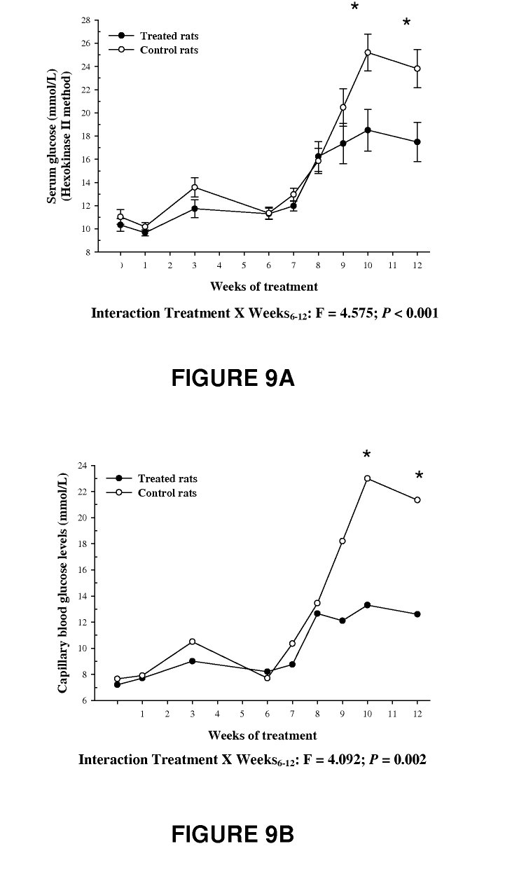

FIG. 9A shows mean serum glucose levels using hexokinase (HK) II method while FIG. 9B shows median capillary blood glucose levels as measured using a glucose meter kit. Glucose levels measured using both methods (FIGS. 9A and 9B) suggest an effect of PDS on glycemia and/or insulin secretion. By week 9 of the treatment, glycemia was stable in controls, and by week 10, it was significantly lower in PDS-treated rats than in control rats (p<0.001 by HK; p=0.002 by glucose meter).

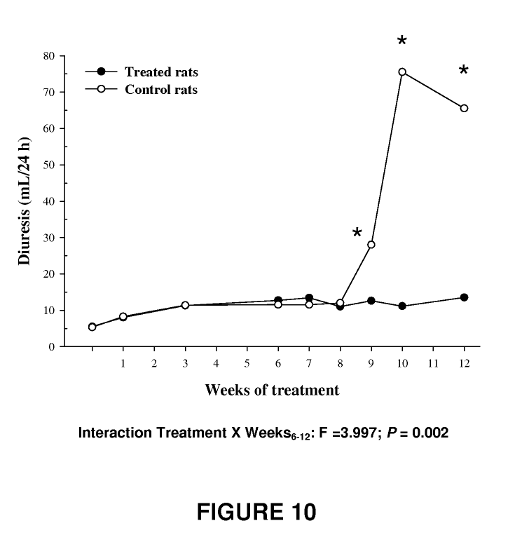

As shown in FIG. 10, diuresis (i.e. urine production) started to increase at week 9 in control animals, in very good correlation with the increase in the glycemia (r=0.888, p<0.001). This is most probably due to the osmotic diuresis consecutive to hyperglycemia. The difference of diuresis between groups (lower in the PDS-treated rats) reflects the difference in glycemia also observed for the same time points.

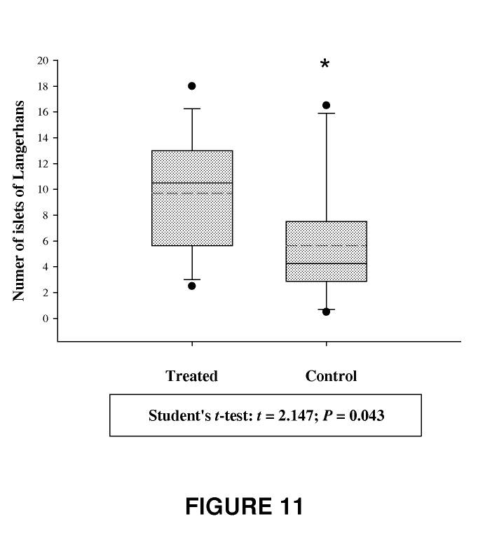

FIG. 11 shows results from histology of the pancreas of control and PDS-treated rats. The pancreas from treated animals displayed a higher number of islets of Langerhans per field, a significant difference from controls that could explain the higher insulin level measured and displayed in FIG. 8. Those histological results suggest that PDS is beneficial in protecting pancreas function and in slowing down the rate of disappearance of the islets.

The effect of the PDS treatment on kidney gene expression was also measured. Briefly, whole kidney RNA was isolated and pooled from 2-4 individual rats from each group (controls and treated group). The polled RNA was then processed using a Gene Chip Rat Exon 1.0 ST Array™ (Affimetrix) according to the manufacturer's standard procedure and analyzed for differential expression between the two groups on gene and exon levels. Although not shown, 75 genes were found to be up regulated and 43 down regulated in the treatment group. The Peroxisome proliferator-activated receptor gamma (PPARG) was among the genes showing up regulation, with a 1.85 fold increase. PPARG is a well known transcriptional regulator that regulates lipid, glucose and amino acid metabolism, and this receptor is the main target of the thiazolidinediones used in diabetes mellitus and other diseases that feature insulin resistance.

Taken together, those results suggests that 1,3 Propanedisulfonic Acid Disodium Salt (PDS) has beneficial effects on glucose metabolism and insulin secretion by decreasing glycemia and increasing insulin secretion and/or increasing insulin sensitivity. These biochemical results are corroborated by the higher number of islets of Langerhans in PDS-treated rats, suggesting a decrease of the rate of exhaustion of the islets, which could explain the higher insulin levels observed. These effects on pancreatic cells and on glucose/insulin levels support the potential medical utility of PDS in the prevention or treatment of various pancreatic diseases where preventing loss of islets of Langerhans or stabilizing their function is desirable, including type 1 and type 2 diabetes.

The effect of PDS of triglyceride levels as well as cholesterol levels combined with the fact that it was shown to have a beneficial effect on glucose levels as well as insulin levels supports its potential use in the treatment of conditions and diseases such as metabolic syndrome or diabetes with features of metabolic syndrome.

### Example 12

### In vivo Preventive Study of Pancreatic Function in SHR Rats

SHR rats are non-diabetic rats but having insulin resistance. Rats are divided in two groups which are administered PDS or vehicle respectively. After initiation of treatment, all rats are administered streptozotocine at low dose with the aim to chemically destroy a portion of Langerhans islets. Both treated and control rats are challenged by an Oral Glucose Tolerance Test (OGTT) and glucose levels are measured. Animals receiving PDS previously experience lower glucose levels than control. Lower glucose levels would suggest an effect in protection of islets of Langerhans as well as a potential use in delaying treatment with insulin in diabetic patients.

### Example 13

### Treatment of Human Patients (Diabetic Nephropathy)

A patient requiring treatment for diabetic neuropathy is treated with 1,3 Propanedisulfonic Acid Disodium Salt (PDS) (800 mg) twice daily. The dose is adjusted by the physician (e.g. maintained, increased to 1200 mg or lowered to 400 mg) according to the patient's response to the treatment as measured by its renal function (e.g. GFR, creatinine clearance, uric acid clearance, albuminuria, etc.).

### Example 14

### Treatment of Human Patients (Diabetes)

A patient requiring treatment for diabetes is treated with 1,3 Propanedisulfonic Acid Disodium Salt (PDS) (800 mg) twice daily. The dose is adjusted by the physician (e.g. maintained, increased to 1200 mg or lowered to 400 mg) according to the patient's response to the treatment as measured by its pancreatic function or its insulin sensitivity (e.g. insulin serum levels, insulin secretory capacity, glycemia, diuresis, etc.).

### Example 15

### Treatment of Human Patients (Hyperlipidemia)

A patient requiring treatment for hyperlipidemia (e.g. hypercholesterolemia, hypertriglyceridemia, hyperlipoproteinemia, etc.) is treated with 1,3 Propanedisulfonic Acid Disodium Salt (PDS) (800 mg) twice daily. The dose is adjusted by the physician (e.g. maintained, increased to 1200 mg or lowered to 400 mg) according to the patient's response to the treatment as measured by its blood lipids levels (e.g. free triglycerides, LDL cholesterol, HDL cholesterol etc.).

### Example 16

### Treatment of Human Patients (Vascular or a Cardiovascular Disease)

A patient requiring treatment for vascular or a cardiovascular disease (e.g. hypertension, arteriosclerosis, atherosclerosis, myocardial infarction, etc) is treated with 1,3 Propanedisulfonic Acid Disodium Salt (PDS) (800 mg) twice daily. The dose is adjusted by the physician (e.g. maintained, increased to 1200 mg or lowered to 400 mg) according to the patient's response to the treatment (e.g. blood pressure).

### Example 17

### Treatment of Human Patients (Diabetes with Features of Metabolic Syndrom)

As previously described, serious side effects are often observed in patients using current diabetes treatment, so that lowering the required dosage of these treatment is highly desirable. In addition, despite treatment many diabetic patients continue to have poorly controlled blood glucose levels and remain at risk of diabetes-related health complications. The presence of metabolic syndrome in these patients may also further these health risks. Therefore, additional treatment with complementary mechanisms of action, including targeting features of metabolic syndrome, would be beneficial to Type 2 diabetic patients. However, given that these patients are already at increased risk for serious complications, any add-on treatment must in addition have favourable safety profile. Compound1,3-propanedisulfonic acid has previously been tested clinically in patients with AA amyloidosis and can be safely used in humans.

Patients with Type 2 diabetes and features of metabolic syndrome receive 1600 mg of PDS (four 400 mg capsules) two times a day (BID). Patients are on stable therapeutic dose of metformin alone or metformin in combination with a sulfonylurea agent for a minimum period of 3months prior to the beginning of treatment with PDS. In addition, patients may be receiving other concomitant medication, such as statins, angiotension converting enzyme (ACE) inhibitors, angiotension receptor blockers (ARB), thiazide diuretics, or β-blockers. Patients' parameters are monitored.

As an example, PDS is administered in combination with metformin or metformin/sulfonylurea dual therapy in inadequately controlled Type 2 diabetic subjects that have HbA1c (glycosylated haemoglobin) levels that range between 7.5% and 10% (inclusively).

Parameters evaluated are changes from baseline in the HbA1c levels, rate of change in HbA1c over the treatment, rate of achieving glycemic control. HbA1c within the red blood cell reflects the average level of glucose to which the cell has been exposed during its normal multi-week life cycle. The HbA12c measure is an appropriate measure of blood glucose, as it is a reliable and accurate.

Other parameters measured include standard tests for the assessment of diabetes and features of metabolic syndrome, including fasting serum glucose, insulin resistance, insulin secretion levels, serum triglycerides, serum insulin, cholesterol (Total, HDL, and LDL), waist circumference, body impedance, microalbuminuria/proteinuria, creatinine clearance, serum creatinine, and blood pressure (systolic and diastolic). Uric acid clearance is also assessed to further demonstrate the potential pharmacological effects of PDS.

1. A method for preventing or treating a pancreatic disorder in a subject in need thereof, comprising administering to said subject an effective amount of a compound of the Formula (I):
Y—(CH₂)ₙ—(CH)ₜ—[CH₂Y]ₘ (I)
wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2.

2. The method of claim 1, wherein the subject has hyperglycemia.

3. The method of claim 1, wherein the pancreatic disorder is diabetes mellitus.

4. The method of claim 3, wherein said diabetes is associated with features of metabolic syndrome.

5. The method of claim 1, wherein said method further comprises administering a second agent.

6. The method of claim 5, wherein said second agent is a biguanide or a sulfonylurea.

7. The method of claim 6, wherein said second agent is metformin.

8. The method of claim 7, wherein the method further comprises administering a sulfonylurea.

9. A method for preventing or treating metabolic syndrome in a subject in need thereof, comprising administering to said subject an effective amount of a compound of the Formula (I):
Y—(CH₂)ₙ—(CH)ₜ—[CH₂Y]ₘ (I)
wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2.

10. A method for preventing or treating diabetes mellitus in a subject in need thereof, comprising administering to said subject an effective amount of a compound of the Formula (I):
Y—(CH₂)ₙ—(CH)ₜ—[CH₂Y]ₘ (I)
wherein Y is SO₃X or OSO₃X independently chosen for each occurrence; X is a cationic group which independently for each occurrence is hydrogen, lithium, sodium, potassium, calcium, magnesium, trialkylammonium or aluminum; n is 1, 2, 3 or 4; t is 0 when m is 1; and t is 1 when m is 2.

11. The method of claim 10, wherein the diabetes mellitus is type 1 diabetes.

12. The method of claim 11, wherein the diabetes mellitus is type 2 diabetes.

13. The method of claim 10, wherein said diabetes is associated with features of metabolic syndrome.

14. The method of claim 10, wherein said method positively affects in said subject at least one pancreatic function parameter which is size, growth and/or secreting activity of islets of Langerhans, size, growth and/or secreting activity of beta-cells; insulin secretion, insulin blood levels, or glucose blood levels.

15. The method of claim 1, wherein said method restores or improves pancreatic function by preventing loss or stimulating neogenesis of islets of Langerhans.

16. The method of claim 14, wherein said pancreatic function is assessed by measuring serum insulin levels, by measuring glycemia, by measuring diuresis, by measuring kalemia, by imaging of the pancreas or by making a histological examination of the pancreas.

17. The method of claim 1, wherein said compound is 1,3-propanedisulfonic acid or a pharmaceutically acceptable salt thereof.

18. The method of claim 1, wherein said compound is 1,3-propanedisulfonic acid disodium salt.

19. The method of claim 1, wherein the subject does not have amyloidosis.

20. The method of claim 1, wherein the subject does not have AA-amyloidosis.

21. The method of claim 1, wherein the subject does not have IAPP-amyloidosis.

22. The method of claim 1, wherein the subject does not have a renal disorder.

23. The method of claim 1, wherein the subject does not have a nephropathy (e.g. diabetic nephropathy).

24. Use of 1,3-propanedisulfonic acid or a pharmaceutically acceptable salt thereof for the prevention or treatment of a pancreatic disorder in a subject in need thereof.

25. Use of 1,3-propanedisulfonic acid or a pharmaceutically acceptable salt thereof for the prevention or treatment of the metabolic syndrome in a subject in need thereof.

26. Use of 1,3-propanedisulfonic acid or a pharmaceutically acceptable salt thereof for the prevention or treatment of diabetes mellitus in a subject in need thereof.

27. Use of 1,3-propanedisulfonic acid or a pharmaceutically acceptable salt thereof for the prevention or treatment of diabetes mellitus with features of metabolic syndrome in a subject in need thereof.

28. A method of preventing or decreasing proteinuria in a subject in need thereof, comprising administering to said subject an effective amount of 1,3-propanedisulfonic acid or a pharmaceutically acceptable salt thereof.

29. A method of increasing insulin secretion and/or increasing insulin sensitivity in a subject in need thereof, comprising administering to said subject an effective amount of 1,3-propanedisulfonic acid or a pharmaceutically acceptable salt thereof.

30. A method of decreasing insulin resistance in a subject in need thereof, comprising administering to said subject an effective amount of 1,3-propanedisulfonic acid or a pharmaceutically acceptable salt thereof.

31. A method of decreasing hyperglycemia in a subject in need thereof, comprising administering to said subject an effective amount of 1,3-propanedisulfonic acid or a pharmaceutically acceptable salt thereof.

32. A method for delaying the requirement for treating a diabetic patient with insulin by administering an effective amount of 1,3-propanedisulfonic acid.
